(19) United States
(12) Patent Application Publication
(10) Pub. No.: US 2006/0030536 A1
(43) Pub. Date: Feb. 9, 2006

(54) Combination therapies for cancer and proliferative angiopathies

(75) Inventors: Hua E. Yu, Glendora, CA (US); Richard Jove, Glendora, CA (US); Jin Q. Cheng, Tampa, FL (US); Guilian Niu, Zephryhills, FL (US); Said Sebti, Tampa, FL (US)

Correspondence Address: H. Lee Moffitt Cancer Center and Research;Institute, Inc., Attn:Jarett Rieger, 12902 Magnolia Drive (MRC-TTO), Tampa, FL, 33612-9497, US

(73) Assignee: University of South Florida, Tampa, FL (US)

(21) Appl. No.: 11/102,911

(22) Filed: Apr. 8, 2005

Related U.S. Application Data
(60) Provisional application No. 60/560,884, filed on Apr. 9, 2004.

(51) Int. Cl. A61K 48/00; A61K 39/395

(52) U.S. Cl. 514/44; 424/155.1

(57) ABSTRACT

Compositions and methods for treating cancer and proliferative angiopathies are provided. A composition can include an inhibitor of the Jak2/Stat3 signaling pathway and an inhibitor of the PI3k/Akt signaling pathway. In certain cases, the two inhibitors are capable of acting synergistically as compared to either inhibitor alone.

### CROSS-REFERENCE TO RELATED APPLICATIONS

This application claims priority under 35 U.S.C. § 119(e)(1) to U.S. Provisional Application Ser. No. 60/560,884 filed Apr. 9, 2004, which is herein incorporated by reference in its entirety.

### STATEMENT AS TO FEDERALLY-FUNDED RESEARCH

Funding for the work described herein was provided in part by the United States federal government, which may have certain rights in the invention.

### TECHNICAL FIELD

Provided herein are compositions that include an inhibitor of a Jak2/Stat3 signaling pathway and an inhibitor of a PI3k/Akt signaling pathway, pharmaceutical compositions including the same, and methods of using such compositions to treat cancer, such as solid and hematological cancers, and proliferative angiopathies.

### BACKGROUND

Vascular endothelial growth factor (VEGF) has a well established role in angiogenesis and tumor progression. Inhibition of VEGF and/or VEGFR signaling has shown promise for tumor anti-angiogenesis therapy in both animal models and cancer patients. A large number of oncoproteins that are activated in cancer cells, however, act as VEGF inducers, creating a challenge for the inhibition of VEGF production. For example, the PI3k/Akt signaling pathway upregulates expression of VEGF in both tumor and endothelial cells, with hypoxic inducible factor-1 (HIF-1) mediating the PI3k/Akt-induced VEGF expression; see, e.g., Semenza G. L. (2003) Nat. Rev. Cancer 3:721-732. In addition to controlling angiogenesis, HIF-1 regulates metabolic adaptation to hypoxia and other critical aspects of tumor progression. HIF-1 consists of two subunits: an inducible HIF-1α subunit, which is frequently upregulated by intratumoral hypoxia and by genetic alterations that activate the PI3k/Akt signaling pathway, and a constitutively expressed HIF-1β subunit.

Signal transducers and activators of transcription (Stats) are latent cytoplasmic transcription factors that function as intracellular effectors of cytokine and growth factor signaling pathways. Constitutive activation of certain Stat family members, such as Stat3, accompanies a wide range of human malignancies, including both hematologic and solid cancers. Recent studies have also identified Stat3 as a direct transcription activator of the VEGF gene. Activation of Stat3 leads to tumor angiogenesis in vivo and blocking Stat3 signaling in tumors can cause reduction of tumor angiogenesis. A role of Stat3 in upregulating VEGF expression in diverse human cancers has also been demonstrated. Importantly, constitutive activation of Stat3 occurs at about 50% to 90% frequency in a broad range of human cancers, suggesting that Stat3 activity contributes significantly to tumor VEGF overproduction.

Breast cancer is the most frequent malignancy in the Western world and the second leading cause of cancer death in women in the United States. One of the most thoroughly studied areas in breast cancer biology is that of the role of a set of receptor tyrosine kinases (RTKs), known as the ErbB family, in breast normal development as well as breast oncogenesis. Mammalian cells express four members of this family: ErbB1 (or HER-1), the receptor for EGF, ErbB2 (or HER-2 or Neu), ErbB3 (or HER-3) and ErbB4 (or HER-4). Dimerization of these receptors promotes stimulation of the intrinsic tyrosine kinase activity, autophosphorylation of a specific tyrosine in the cytoplasmic domain of the receptors and recruitment of signaling proteins that trigger a variety of complex signal transduction pathways. ErbB is known to be overexpressed in many human breast cancer cell lines. Activation of these receptors either by overexpression- or ligand-induced dimerization results in the activation of at least three major oncogenic and tumor survival pathways, leading to high levels of phosphorylated forms of the serine/threonine kinases Akt and Erk, as well as the signal transducer and activator of transcription, Stat3.

Jak2/Stat3 and PI3k/Akt are two parallel pathways that mediate the functions of many receptor and non-receptor tyrosine kinases, including EGFR (ErbB1), Her-2 (ErbB2), and c-Src. IL-6R, which is frequently activated in cancers, also signals through both Jak2/Stat3 and PI3k/Akt pathways. Overexpression and/or persistent activation of EGFR/Her-2, Src and IL-6R are known to promote tumor growth/survival and to induce VEGF expression and angiogenesis. IL-6R activity also activates the PI3k/Akt pathway. Interestingly, it has been shown that blocking of Stat3 signaling, but not of PI3k/Akt signaling, inhibits VEGF expression in tumor cells with constitutive IL-6R signaling, suggesting that Stat3 can continue to activate VEGF expression in the absence of PI3k/Akt signaling.

Several approaches have been taken to inhibit ErbB1 and ErbB2 overexpression including antibodies against the extracellular portions of ErbB1 (i.e. Erbitux, C-225) and ErbB2 (i.e. Herceptin, trastuzamab), as well as inhibitors of their tyrosine kinase activities (i.e. Iressa for ErbB1). Inhibitors of the downstream signal transduction pathways activated by the ErbB family members have been designed, including inhibitors of PI3k (LY294002) and Mek (PD184352). A more recently identified Jak2/Stat3 signaling inhibitor, JSI-124, does not inhibit the PI3k/Akt or Mek/Erk pathways.

### SUMMARY

Provided herein are materials and methods for treating cancer and proliferative angiopathies. For example, compositions and articles of manufacture are provided that include 1) an inhibitor of the PI3k/Akt signaling pathway; and 2) an inhibitor of the Jak2/Stat3 signaling pathway. A synergistic effect on tumor cell growth inhibition and programmed tumor cell death can occur when both the PI3k/Akt and Jak2/Stat3 pathways are inhibited.

In some cases, an inhibitor can be selective for a particular pathway, such as by inhibiting a member of the pathway or by inhibiting a protein that selectively activates one pathway. As used herein, “selective” for a particular pathway means that an inhibitor preferentially or exclusively inhibits that pathway relative to the other pathway. In other cases, one inhibitor can inhibit both pathways. In such cases, the second inhibitor for inclusion in a composition or for use in a method described herein should be chosen to selectively inhibit only one of the pathways. Thus, for example, Herceptin can be used to inhibit both PI3k/Akt and Jak2/Stat3 pathways; a second inhibitor for use with Herceptin can be a selective AKT inhibitor or a selective STAT3 inhibitor, e.g., small-molecule inhibitors that bind noncovalently to AKT or to STAT3. Methods for using the compositions and articles of manufacture to inhibit tumor cell growth and angiogenesis and to treat cancers and proliferative angiopathies are also provided.

Accordingly, in one embodiment, a composition of matter or article of manufacture including:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof, is provided.

Any combination of inhibitors can be used. An inhibitor of the Jak2/Stat3 signaling pathway can inhibit a protein that activates Jak2. An inhibitor of the Jak2/Stat3 signaling pathway may not, in some cases, inhibit the PI3k/Akt signaling pathway. An inhibitor of the PI3k/Akt signaling pathway can inhibit a protein that activates PI3k. An inhibitor of the PI3k/Akt signaling pathway, in some cases, may not inhibit the Jak2/Stat3 signaling pathway.

In some embodiments, an inhibitor of the Jak2/Stat3 signaling pathway inhibits Jak2 or Stat3. For example, an inhibitor of Jak2 or Stat3 can reduce the expression level of the Jak2 protein or Stat3 protein, respectively, in a cell. An inhibitor of Jak2's or Stat3's expression level can be an isolated nucleic acid that, when transcribed in a cell, results in an siRNA, a ribozyme, or an antisense nucleic acid. In other cases, an inhibitor of Jak2's or Stat3's expression level is an siRNA nucleic acid or antisense nucleic acid.

An inhibitor of Jak2 can inhibit an activity of Jak2, such as a kinase activity. An inhibitor of Jak2 can bind noncovalently to Jak2, e.g., an antibody or antibody fragment or a small molecule.

An inhibitor of Stat3 can inhibit an activity of Stat3. Stat3 activity can be Stat3 dimerization, Stat3 DNA binding, or Stat3 transactivation. An inhibitor of Stat3 can bind noncovalently to STAT3, e.g., an antibody or antibody fragment, or a small-molecule, such as CPA-1 or CPA-7.

An inhibitor of the PI3k/Akt pathway can inhibit PI3k. In some cases, an inhibitor of PI3k reduces the expression level of the PI3k protein in a cell. An inhibitor of PI3k can inhibit an activity of PI3k, such as a kinase activity. An inhibitor of PI3k can bind noncovalently to PI3k.

An inhibitor of the PI3k/Akt pathway can inhibit Akt, e.g., by reducing the expression level of the Akt protein in a cell or by inhibiting an activity of Akt, such as a kinase activity. An inhibitor of Akt can bind noncovalently to Akt, such as the small-molecule TCN.

In another embodiment, pharmaceutical compositions are provided. A pharmaceutical composition can include any of the compositions and/or inhibitors described herein, and a pharmaceutically acceptable carrier. A composition, article of manufacture, or pharmaceutical composition can be used for the treatment, prevention, or amelioration of one or more symptoms of cancer or a proliferative angiopathy. A composition, article of manufacture, or pharmaceutical composition can be used in the manufacture of a medicament for the therapeutic and/or prophylactic treatment of cancer or a proliferative angiopathy.

In another aspect, a method for treating, preventing, or ameliorating one or more symptoms of cancer or a proliferative angiopathy in a mammal is provided, which includes administering:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof to the mammal.

A mammal can be any mammal, including a human. A cancer can be a solid or hematologic cancer, e.g., breast, prostate, melanoma, multiple myeloma, leukemia, pancreatic, ovarian, head and neck, and brain cancers. A proliferative angiopathy can be diabetic microangiopathy. Any combination of inhibitors can be used. In certain cases, two small-molecule inhibitors specific for protein members of the pathways are used, e.g., a small-molecule inhibitor or Jak2 or Stat3 and a small-molecule inhibitor of PI3k or Akt. In certain cases, the two inhibitors are capable of acting synergistically to treat, prevent, or ameliorate said one or more symptoms as compared to either inhibitor alone.

In yet another aspect, provided herein is a method for inhibiting the growth of a cancer cell. The method can include contacting a cancer cell with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof. The inhibitor of the Jak2/Stat3 signaling pathway and the inhibitor of the PI3k/Akt signaling pathway can be capable of acting synergistically to inhibit the growth of said cancer cell as compared to either inhibitor alone.

Also provided is a method for inducing apoptosis in a cancer cell that includes contacting the cancer cell with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof. The inhibitor of the Jak2/Stat3 signaling pathway and the inhibitor of the PI3k/Akt signaling pathway can be capable of acting synergistically to induce apoptosis in the cancer cell as compared to either inhibitor alone.

In yet another aspect, a method of inhibiting angiogenesis from a cancer tumor is provided. The method includes contacting the cancer tumor with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof. Contacting can be by any means. Any combination of inhibitors can be used. In certain cases, the two inhibitors are small-molecule inhibitors of protein members of both pathways, e.g., a small molecule inhibitor of Jak2 or Stat3 and a small-molecule inhibitor of PI3k or Akt.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A demonstrates that MCF-7 breast cancer cells treated with IL-6 at the indicated concentrations had elevated expression of HIF-1α—but not HIF-1β—protein. Nuclear proteins were used for the Western blot analysis.

FIG. 1B demonstrates that IL-6 at 20 ng/ml increases levels of both total and activated AKT proteins. The antibody used for detecting phospho-AKT (PAKT) by Western blot recognizes both AKT1 and AKT2. For the total AKT protein detection, the antibody is specific for AKT1. An increase in HIF-1α and VEGF protein levels was also detected in the nuclear and cytoplasmic proteins, respectively, prepared from the same cells.

FIG. 1C demonstrates that IL-6 induces Stat3 DNA-binding/activity in MCF-7 cells as determined by EMSA.

FIG. 2A is a Northern blot analysis of HIF-1α mRNA levels in MCF-7 tumor cells treated with IL-6 at the indicated concentrations. Ribosomal RNAs (28s and 18S) are internal controls for RNA loaded in each lane.

FIG. 2B is a Western blot showing inhibition of protein synthesis by cycloheximide (CHX); the blot indicates a reduction of HIF-1α protein with time. 20 ng/ml of IL-6 was used.

FIG. 2C is an SDS-PAGE of a pulse-label assay of HIF-1α immunoprecipitates. 20 ng/ml of IL-6 was used. Imaging quantification of the HIF-1α bands, labeled 1-4, is expressed in arbitrary units.

FIG. 3A is a Western blot analysis of HIF-1α and VEGF protein levels in control empty vector-transfected and siRNA/Stat3 expressing MCF-7 tumor cells (top panel). In these experiments, nuclear protein was used for detection of HIF-1α and cytoplasmic proteins from the same cells were analyzed for VEGF expression levels. A considerable reduction in Stat3 DNA-binding activity, as determined by EMSA, was seen in siRNA/Stat3 MCF-7 cells compared to the control MCF-7 cells (bottom panel).

FIG. 3B: The top panel is a Western blot analysis and the bottom panel is an EMSA demonstrating a requirement for Stat3 signaling in both the basal and IEL-6-induced HIF-1α expression is confirmed in MEFs. 20 ng/ml IL-6 was used in these experiments.

FIG. 4A demonstrates that treating A2058 human melanoma cells with Src tyrosine kinase inhibitors, PD166285 or PD180970, resulted in reduction of HIF-1α expression, as shown by Western blot analysis (top panel) and Stat3 DNA-binding activity, as shown by EMSA (bottom panel).

FIG. 4B demonstrates that blocking Stat3 signaling by siRNA in the A2058 tumor cells decreased the expression of both HIF-1α and VEGF proteins. A decrease in Stat3 DNA-binding in the siRNA/Stat3 A2058 tumor cells is shown by EMSA in the right panel.

FIG. 5A is a Western blot demonstrating that Heregulin upregulates HIF-1α expression in MCF-7 breast cancer cells.

FIG. 5B demonstrates increased Stat3 DNA-binding activity by heregulin in MCF-7 by EMSA.

FIG. 5C shows that HIF-lIa and VEGF upregulation by Her-2 activation requires Stat3. Western blot analysis of control vector and siRNA/Stat3-transfected MCF-7 cells showed a requirement for Stat3 in both basal and Her-2-induced HIF-1α and VEGF upregulation.

FIGS. 6A and B show that targeting Stat3 by small-molecule Stat3 inhibitors reduces HIF-1α and VEGF expression in tumor cells. Treatment of DU145 prostate cancer cells with either ISS CPA7 (A) or IS3 295 (B) resulted in lowered Stat3 DNA-binding activity (bottom panel, EMSAs) and expression of both HIF-1α and VEGF proteins (top panel, Western blots).

FIG. 7 is a Western blot analysis of protein samples prepared from MCF-7 human breast cancer cells transfected with either a control vector or the siRNA/Stat3 expression vector as indicated (left panel). MEFs with or without the Stat3 alleles were also subjected to Western blot analysis (right panel).

FIG. 8 demonstrates tumor angiogenesis as determined by Matrigel assays. Left, photos of indicated Matrigel plugs harvested from mice five days after implantation. Right, quantification of hemoglobin contents in the Matrigels. For each group, n=4.

FIGS. 9A-9F demonstrate the effect of LY 294002 and JSI-124, either alone or in combination, on cell proliferation. Human breast cancer MDA-MB-468 (A), MDA-MB-231 (B) and MCF-7 (C) cell lines were grown in a 96-well plate. At -50% confluence, cells were treated with either DMSO or 1, 5, 10, and 40 μM LY294002 and 0.01, 0.05, 0.1, 0.5, and 1 μM JSI-124 in combination or alone for 60 h.; cells were then subjected to MTT assay and synergistic effects between two drugs were determined and plotted as shown in Isoblogram. Similar results were observed in three independent experiments for FIG. 9 A and B and in two independent experiments for FIG. 9C.

FIG. 10 demonstrates the induction of tumor cell death in MDA-MB-468 cells with treatment by LY294002 and JSI-124, either alone or in combination. MDA-MB-468 cells were treated with vehicle DMSO (control), 10 or 20 μM LY294002; 0.05 EM JSI-124; 10+0.05 μM LY294002+JSI-124; or 20+0.05 μM LY294002+JSI-124 for 48 h, followed by trypan blue dye exclusion assay. The numbers indicate the percentage of dead cells. Standard deviations are shown with error bars. Similar results were observed in another independent experiment.

FIG. 11 demonstrates the induction of apoptosis in MDA-MB-468 cells with treatment by LY294002 and JSI-124, either alone or in combination. MDA-MB-468 cells were treated with vehicle DMSO (control), 20 μM LY294002, 0.1 or 0.05 μM JSI-124 as single agents. Combination treatment consisted of 20+0.1 or 20+0.05 μM LY294002+JSI-124 for 48 h, followed by Tunel analysis. The numbers indicate the percentage of TUNEL-positive population. The result of one independent experiment is shown here.

FIGS. 12A and 12B show that JSI-124 and LY294002 act synergistically to decrease the levels of the pro-survival protein Bc1-XL and to induce PARP cleavage. MDA-MB-468, MDA-MB-453 and MCF-7 breast cancer cells were treated with vehicle DMSO (control), 20 Mμ L (LY294002), 0.5 Mμ J (JSI-124) or 20+0.05 Mμ L+J for 48 h, followed by Western blot assay using specific antibodies to Bc1-xL, PARP and actin (internal control).

FIG. 13 shows the effect of LY294002 and JSI-124, either alone or in combination, on cell cycle progression. MDA-453 cells were treated with vehicle DMSO (control), 20 μM LY294002; 0.05 μM JSI-124 or 20+0.05 μM LY294002+JSI-124 for 48 h, followed by flow cytometry analysis.

FIG. 14 shows the structure of naltrindole.

FIG. 15 shows the structures of a variety of peptidomimetics useful for STAT3 DNA-binding inhibition.

FIG. 16 shows the structures of some platinum(IV) complexes useful for STAT3 DNA-binding inhibition.

FIG. 17 shows the structures of some Src kinase inhibitors.

FIG. 18 demonstrates that inhibition of Stat3 results in an inhibition of the expression of the protein Surviving.

### DETAILED DESCRIPTION

The term “expression” refers to the process of converting genetic information encoded in a gene or polynucleotide into RNA (e.g., mRNA, rRNA, tRNA, or snRNA) through “transcription” of the gene or polynucleotide (i.e., via the enzymatic action of an RNA polymerase), and into protein, through “translation” of mRNA. Expression may be regulated at many stages in the process.

As used herein, an “isolated nucleic acid” refers to a nucleic acid that is separated from other nucleic acid molecules that are present in a genome, including nucleic acids that normally flank one or both sides of the nucleic acid in a genome. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. The term “isolated” as used herein with respect to nucleic acids also includes any non-naturally-occurring nucleic acid sequence since such non-naturally-occurring sequences are not found in nature and do not have immediately contiguous sequences in a naturally-occurring genome. A nucleic acid existing among hundreds to millions of other nucleic acids within, for example, cDNA libraries or gendmic libraries, or gel slices containing a genomic DNA restriction digest, is not to be considered an isolated nucleic acid.

Nucleic acids of the invention can be in a sense or antisense orientation, can be complementary to a reference sequence, e.g., in a sequence listing, and can be DNA, RNA, or nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7: 187-195; Hyrup et al., 1996, Bioorgan. Med. Chem., 4: 5-23. In addition, the deoxyphosphate backbone can be replaced with, for example, a phosphorothioate or phosphorodithioate backbone, a phosphoroamidite, or an alkyl phosphotriester backbone.

Isolated nucleic acid molecules can be produced by standard techniques. For example, polymerase chain reaction (PCR) techniques can be used to obtain an isolated nucleic acid containing a nucleotide sequence described herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length (e.g., 10, 15, 20, 25, 27, 34, 40, 45, 50, 52, 60, 65, 70, 75, 82, 90, 102, 150, 200, 250 nucleotides in length). General PCR techniques are described, for example in PCR Primer: A Laboratory Manual, Ed. by Dieffenbach, C. and Dveksler, G., Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis, 1992, Genetic Engineering News, 12: 1; Guatelli et al., 1990, Proc. Natl. Acad. Sci. USA, 87: 1874-1878; and Weiss, 1991, Science, 254: 1292.

Isolated nucleic acids of the invention also can be chemically synthesized, either as a single nucleic acid molecule (e.g., using automated DNA synthesis in the 3′ to 5′ direction using phosphoramidite or phosphorothioate technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids of the invention also can be obtained by mutagenesis. For example, a reference nucleic acid sequence be mutated using standard techniques including oligonucleotide-directed mutagenesis and site-directed mutagenesis through PCR. See, Short Protocols in Molecular Biology, Chapter 8, Green Publishing Associates and John Wiley & Sons, Edited by Ausubel, F. M et al., 1992.

The term “polypeptide” refers to a chain of at least three amino acid residues (e.g., a chain having 4-20, 20-100, 100-150, 150-200, 200-300, 300-400, 400-500, 500-600, 600-700 residues, or even more residues). The terms polypeptide and protein may be used interchangeably herein. In some cases, a polypeptide can include a phosphorylated tyrosine.

The term “isolated” with respect to a polypeptide refers to a polypeptide that has been separated from cellular components that naturally accompany it. Typically, the polypeptide is isolated when it is at least 60% (e.g., 70%, 80%, 90%, 95%, or 99%), by weight, free from proteins and naturally occurring organic molecules that may be naturally associated with it. In general, an isolated polypeptide will yield a single major band on a reducing and/or non-reducing polyacrylamide gel. In some cases, an isolated polypeptide is chemically synthesized.

Isolated polypeptides can be obtained, for example, by extraction from a natural source (e.g., plant tissue), chemical synthesis, or by recombinant production in a host plant cell. To recombinantly produce polypeptides, a nucleic acid sequence containing a nucleotide sequence encoding the polypeptide of interest can be ligated into an expression vector and used to transform a bacterial, eukaryotic, or plant host cell (e.g., insect, yeast, mammalian, or plant cells). In bacterial systems, a strain of Escherichia coli such as BL-21 can be used. Suitable E. coli vectors include the pGEX series of vectors that produce fusion proteins with glutathione S-transferase (GST). Depending on the vector used, transformed E. coli are typically grown exponentially, then stimulated with isopropylthiogalactopyranoside (IPTG) prior to harvesting. In general, expressed fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety. Alternatively, 6× His-tags can be used to facilitate isolation.

As used herein, pharmaceutically acceptable derivatives of a composition include salts, esters, enol ethers, enol esters, acetals, ketals, orthoesters, hemiacetals, hemiketals, acids, bases, solvates, hydrates or prodrugs thereof Such derivatives may be readily prepared by those of skill in this art using known methods for such derivatization. The compositions produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylaamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a composition with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which a pathway described herein is implicated.

As used herein, amelioration of the symptoms of a particular disorder by administration of a particular composition or pharmaceutical composition refers to any lessening, whether permanent or temporary, lasting or transient that can be attributed to or associated with administration of the composition.

Compositions and Articles of Manufacture

Provided herein are compositions of matter and articles of manufacture. A composition of matter or article of manufacture can include two inhibitors: (a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof.

In a composition of matter, the two inhibitors can be provided in one formulation, such as a pharmaceutically acceptable formulation, e.g., as a mixture. A mixture need not be a homogenous mixture. Thus, the two inhibitors can be, without limitation, separate phases (e.g., oil/water; liquid/solid) or unmixed powders. The relative dosages and amounts of the two inhibitors can vary according to the nature of the inhibitors, the patient's health, the type of illness to be treated, etc. In an article of manufacture, the two inhibitors can be provided as a mixture, as described previously, or provided separately, e.g., in separate vials, needles, ampoules, etc., at dosage levels and amounts that can vary similarly. An article of manufacture can include auxiliary items such as needles, syringes, package inserts, labels, and directions for administration of the inhibitors.

An inhibitor of a PI3k/Akt signaling pathway or an inhibitor of a Jak2/Stat3 signaling pathway can inhibit any protein member of the respective pathway, e.g., PI3k or Akt with respect to the PI3k/Akt pathway and JAK2 or STAT3 with respect to the Jak2/Stat3 pathway.

In some cases, an inhibitor of a PI3k/Akt signaling pathway can inhibit a protein that activates the PI3k/Akt pathway. For example, receptor tyrosine kinases (e.g., EGFr, Her-2) and nonreceptor tyrosine kinases (e.g., Src, Bcr-Ab1) activate the PI3k/Akt pathway by phosphorylating PI3k. Similarly, in some cases, an inhibitor of a Jak2/Stat3 signaling pathway can inhibit a protein that activates the Jak2/Stat3 pathway. For example, receptor tyrosine kinases (e.g., EGFr, Her-2) and nonreceptor tyrosine kinases (e.g., Src, Bcr-Ab1) activate the Jak2/Stat3 signaling pathway by phosphorylating JAK2. In some cases, a protein that activates one or the other of the two pathways can be a protein that preferentially or selectively activates one of the pathways over the other of the pathways.

In other cases, a protein that activates one pathway can activate both pathways. For example, certain receptor tyrosine kinases (EGFr, Her-2) and nonreceptor tyrosine kinases (e.g., Src, Bcr-Ab1) can activate both PI3k/Akt and Jak2/Stat3 pathways. In cases where an inhibitor of a protein that can activate both pathways is used as one inhibitor herein, then a second inhibitor for use herein should selectively inhibit either the PI3k/Akt pathway or the Jak2/Stat3 pathway. For example, a second inhibitor could selectively inhibit a member of one of the pathways, such as PI3k, or Akt, or Jak2, or Stat3, as described below.

With respect to either pathway, inhibition can occur through mechanisms that affect a protein's expression level or a protein's activity. A protein's activity can include, without limitation, kinase activity, dimerization, DNA-binding, or transactivation. Inhibition can occur through a reduction of the level of a protein that normally would be available to function in or to activate a pathway, such as by binding of a protein by an antibody specific for it or by employing antisense, siRNA, or ribozyme technologies to reduce the level of mRNA coding for the protein. In other cases, inhibition can occur through an inhibition of a protein activity itself, such as by binding of a protein by an antibody, inhibition of dimerization of a protein, inhibition of a kinase activity of protein, inhibition of DNA binding of a protein, or inhibition of transactivation of a protein. As used herein, an inhibitor does not include mutant (e.g., dominant negative mutants) of protein members of either pathway or of proteins that activate either pathway.

An inhibitor of a PI3k/Akt signaling pathway can inhibit any protein member of the pathway, such as PI3k or AKT. An inhibitor of the PI3k/Akt signaling pathway can inhibit a protein that activates the PI3k/Akt pathway. For example, receptor tyrosine kinases (e.g., EGFr and Her-2) and non-receptor tyrosine kinases (Src, Bcr-Ab1) activate the PI3k/Akt pathway by phosphorylating PI3k. In some cases, an inhibitor of the PI3k/Akt signaling pathway does not inhibit the Jak2/Stat3 signaling pathway, e.g., is selective for the PI3k/Akt pathway.

An inhibitor of the PI3k/Akt pathway can inhibit PI3k. For example, an inhibitor of PI3k can reduce the expression level of the PI3k protein in a cell. Such an inhibitor can be an isolated nucleic acid that, when transcribed in a cell, results in an siRNA, a ribozyme, or an antisense nucleic acid. For example, a resultant siRNA nucleic acid can be sufficiently specific to the mRNA encoding PI3k to cleave it through RNAi. In other cases, siRNA nucleic acids and antisense nucleic acids can be isolated nucleic acids that can be contacted directly with a cell and that do not need to be transcribed. Additional information on the design of such nucleic acids is provided below and elsewhere.

In some cases, an inhibitor of PI3k inhibits an activity of PI3k. A PI3k activity can be lipid kinase activity. Kinase activity, including lipid kinase activity, Ser/Thr kinase activity, and Tyr Kinase activity, can be evaluated using methods known to those having ordinary skill in the art; a variety of commercially available kits to measure kinase activity can also be employed (e.g., fluorescence assays available from Invitrogen, Perkin Elmer, and others).

An inhibitor of PI3k can bind noncovalently to PI3k. Noncovalent binding can be assessed using a number of analytical techniques well known to those of ordinary skill in the art, including competitive assays with known binders, surface plasmon resonance techniques, etc. In some cases, a noncovalent binder to PI3k can be an antibody or antibody fragment, as discussed more fully below.

An inhibitor of PI3k can be a small-molecule. For example, LY294002 is a small molecule PI3k inhibitor. LY294002 has the chemical name 2-(4-morpholinyl)-8-phenyl-4H-1-benzopyran-4-one). See the Examples below for additional information on LY294002. Wortmiannin can also be used as a small-molecule inhibitor of PI3k.

An inhibitor of the PI3k/Akt pathway can inhibit Akt. For example, an inhibitor of Akt can reduce the expression level of the Akt protein in a cell. Such an inhibitor can be an isolated nucleic acid that, when transcribed in a cell, results in an siRNA, a ribozyme, or an antisense nucleic acid. In other cases, an Akt inhibitor is an isolated nucleic acid that is an siRNA or antisense nucleic acid that does not require transcription in the cell. Additional information on the design of such nucleic acids is provided below and elsewhere.

In some cases, the inhibitor of Akt inhibits an activity of Akt. An Akt activity can be Ser/Thr kinase activity. An inhibitor of Akt can bind noncovalently to Akt. Noncovalent binding can be assessed as described previously and elsewhere. In some cases, a noncovalent binder to Akt can be an antibody or antibody fragment, as discussed more fully below.

In some cases, an inhibitor of Akt can be a small-molecule. A variety of small-molecules that inhibit Akt have been identified. For example, API-2/TCN is an Akt activation inhibitor that is highly selective for Akt and does not inhibit the activation of PI3k, Pdk1, Pkc, Sgk, Pka, Stat3, Erk-½, or Jnk. API-2 (NCI identifier: NSC 154020) is also known as triciribine, tricyclic nucleoside, TCN, and 6-Amino-4-methyl-8-(β-D-ribofuranosyl)-4H,8H-pyrrolo[4,3,2-de]pyrimido[4,5-c]pyridazine.

An inhibitor of Akt can bind noncovalently to a PH-domain of Akt. In some cases, an inhibitor that binds noncovalently to a PH-domain of Akt can inhibit Akt kinase activity. Akti-½, Akti-1, and Akti-2 are small-molecules that inhibit Akt and are thought to bind noncovalently to the PH-domain of Akt. Their structures are as follows:

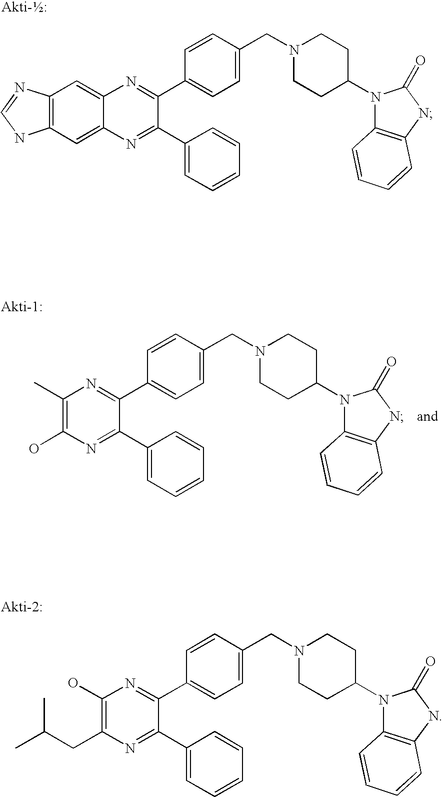

Perifosine, also known as ODPP (octadecyl-(1,1-dimethyl piperidino-4-yl)phosphate), is an alkylphospholipid which competes with phosphatidylino-3,4,5-triphosphate for binding to the PH-domain of Akt. Its structure is shown below:

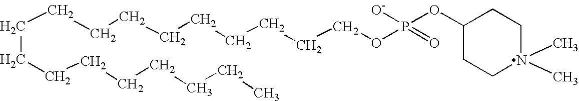

2(R)-2-O-methyl-3-O-octadecylcarbonate (a PIP3 analog) and D-3-deoxy-phosphatidyl-myo-inositols also similarly inhibit Akt and bind noncovalently to Akt. D-3-deoxy-phosphatidyl-myo-inositols (DPIs) cannot be phosphorylated on the 3-position of the myo-inositol ring and include DPI 1-[(R)-2,3-bis(hexadecanoyloxy)propyl hydrogen phosphate], its ether lipid derivative DPI 1-[(R)-2-methoxy-3-octadecyloxypropyl hydrogen phosphate] (DPIEL), and its carbonate derivative DPI 1-[(R)-2-methoxy-3-octadecyloxypropyl carbonate].

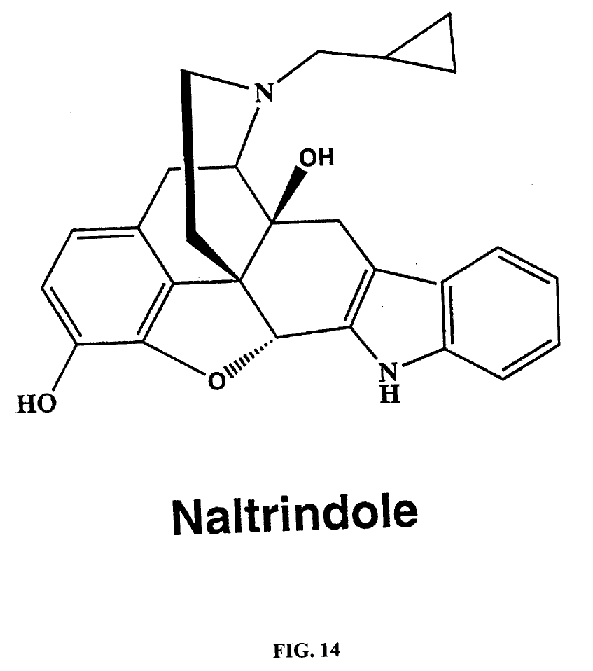

Other small-molecule inhibitors of Akt are also known. Naltrindole is an inhibitor of Akt that binds noncovalently to Akt. Naltrindole has been used as a classic δ opioid antagonist and has the structure set forth in FIG. 14. The plant-derived pigmnent cucurmin and 1L-6-hydroxy-methyl-chiro-inositol are additional examples of Akt inhibitors.

An inhibitor of a Jak2/Stat3 signaling pathway can inhibit any protein member of the pathway, such as Jak2 or Stat3. An inhibitor of the Jak2/Stat3 signaling pathway can inhibit a protein that activates the Jak2/Stat3 pathway. For example, receptor tyrosine kinases (e.g., EGFr and Her-2), non-receptor tyrosine kinases (e.g., Src, Bcr-Ab1), and IL-6 receptor gp130 can activate the Jak2/Stat3 pathway by phosphorylating Jak2.

An inhibitor of a protein that activates the Jak2/Stat3 pathway can, in some cases, also inhibit the PI3k/Akt pathway. For example, the Src tyrosine kinase small-molecule inhibitors, PD166285 and PD180970, (which are known as pyrido[2,3-d]pyrimidine kinase inhibitors) and SU6656 (2-oxo-3-(4,5,6,7-tetrahydro-1 H-indol-2-ylmethylene)-2,3-dihydro-1H-indole-5-sulfonic acid dimethylamide) inhibit the Jak2/Stat3 pathway and the PI3k/Akt pathway.

In some cases, an inhibitor of a protein that activates the Jak2/Stat3 pathway can selectively inhibit the Jak2/Stat3 pathway; e.g., does not inhibit the PI3k/Akt pathway. For example, the small-molecule JSI-124 (Cucurbitacin I, NSC 521777; see structure below) specifically inhibits Ja2/Stat3 activation.

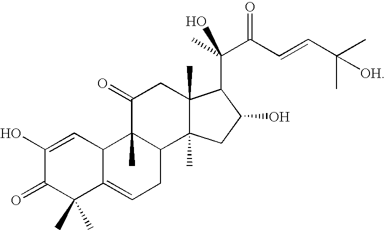

Cucurbitacin B (NSC 49451), E (NSC 106399), and I (NSC 521777) are also selective small-molecule inhibitors of the Jak2/Stat3 pathway. Cucubitacin B, E, and I are known to suppress both Stat3 and Jak2 activation; see, e.g., Sun et al., Oncogene (2005): 1-10 and Blakovich et al., Cancer Res. 63:1270-1279 (2003).

An inhibitor of the Jak2/Stat3 pathway can inhibit Stat3. For example, an inhibitor of Stat3 can reduce the expression level of the Stat3 protein in a cell. Such an inhibitor can be an isolated nucleic acid that, when transcribed in a cell, results in an siRNA, a ribozyme, or an antisense nucleic acid. An antisense or siRNA nucleic acid can also be an isolated nucleic acid that need not be transcribed; e.g., an exogenous sequence for direct administration. For example, the antisense nucleic acid (5′-AAAAAGTGCCCAGATTGCCC-3′; SEQ ID NO:1) was used in the Examples to knock down the expression levels of Stat3. Similarly, the siRNA Stat3 oligonucleotide, AATTAAAAAAGTCAGGTTGCTGGTCAAATTCTCTTGAAATTTGACCAGCAAC CTGACTTCC (SEQ ID NO:2), was used in the Examples to knockdown the expression levels of STAT3.

In some cases, the inhibitor of Stat3 inhibits an activity of Stat3. A Stat3 activity can be, without limitation, dimerization of Stat3 monomers, DNA-binding of Stat3 homodimers, (e.g., to a high-affinity Sis-Inducible Element, hSIE), and transactivation of nucleic acid sequences operably linked to promoters to which Stat3 binds (e.g., promoters of the VEGF gene, BCL-X gene, MCL-1 gene, CYCLIND1 gene, SURVIVIN gene, CD46 gene, and C-MYK gene ). Stat3 is also known to represses and downregulate the proteins P53 and RANTES. More than one Stat3 activity can be inhibited, e.g., dimerization and DNA-binding can both be inhibited by an inhibitor.

An activity of Stat3 can be evaluated using methods known t6 those having ordinary skill in the art. For example, DNA-binding activity of Stat3 homodimers can be assessed using EMSA, as shown in the Examples, below. Dimerization of Stat3 monomers can be assessed using, without limitation, standard competitive binding assays and other protein-protein interaction assays, including FRET assays. Transactivation of a particular gene can be analyzed by expression profiling of the gene under inhibitory and non-inhibitory conditions.

An inhibitor of Stat3 can bind noncovalently to Stat3. Noncovalent binding can be assessed using a number of analytical techniques well known to those of ordinary skill in the art, including competitive assays with known binders, surface plasmon resonance techniques, FRET etc. In some cases, a noncovalent binder to Stat3 can be an antibody or antibody fragment, as discussed more fully below. Certain antibodies to Stat3 are set forth in the Examples.

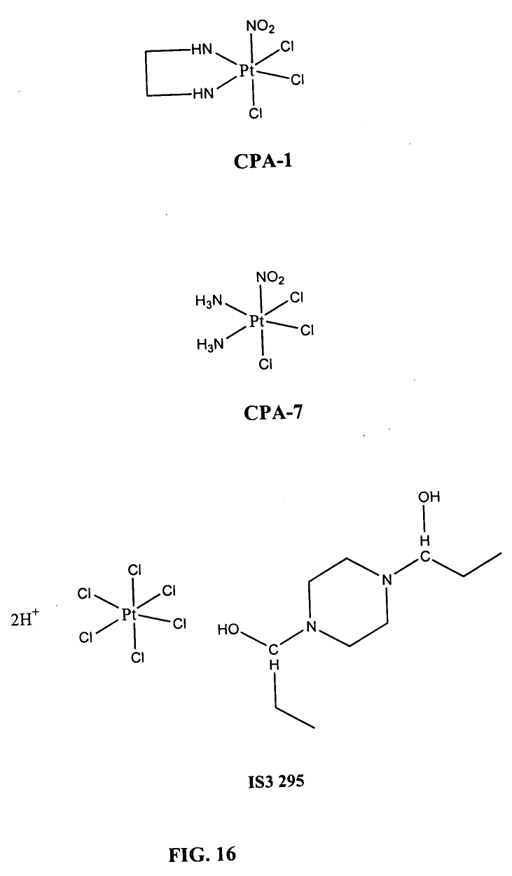

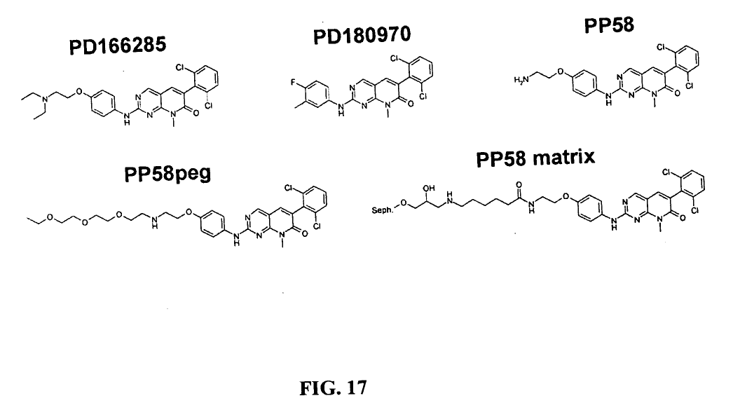

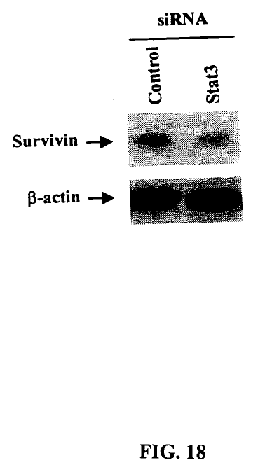

An inhibitor of Stat3 can be a small-molecule. For example, platinum (IV) complexes, which are known to be DNA alkylators, can inhibit Stat3 DNA binding and Stat3 monomer phosphorylation (and thus dimerization) at certain tyrosine residues. Examples of such platinum(IV) complexes include: Pt(IV)Cl₄; CPA-1; and CPA-7 (see FIG. 16 for structures). Other small-molecules that are Stat3 inhibitors include IS3 295 (NSC 295558; see FIG. 16), which inhibits Stat3 DNA binding. In some cases, a small-molecule inhibitor of Stat3 can bind noncovalently to Stat3.

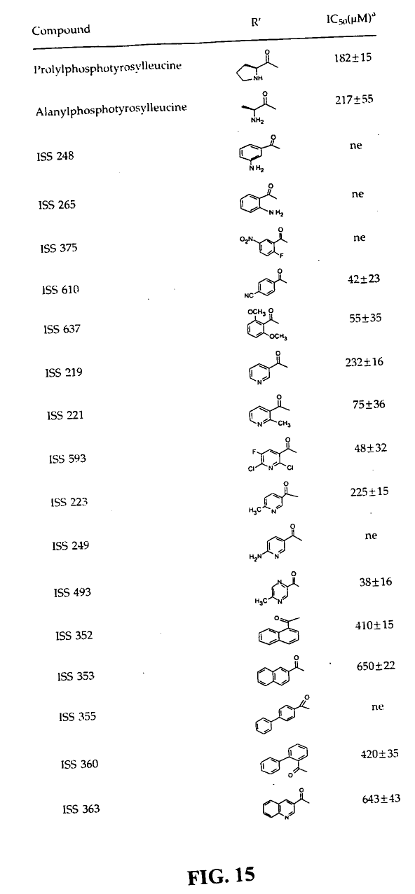

Phosphorotyrosyl-containing peptide molecules have also been shown to be Stat3 inhibitors and to interrupt activated Stat3 dimerization at the SH2 domain, ultimately also leading to reduced DNA binding activity. Phosphorotyrosyl-containing peptides and peptidomimetics thereof can disrupt SH2-domain-phosphorylated tyrosine interactions between phosphorylated STAT3 monomers that lead to dimerization. Examples of such molecules include PY*LKTK (SEQ ID NO:3); PY*LKTK-AAVLLPVLLAAP (SEQ ID NO:4) (which contains a membrane translocating sequence for membrane permeability); PY*L (SEQ ID NO:5), and AY*L (SEQ ID NO:6); in all such sequences a Y* is representative of a phosphorylated tyrosine. Peptidomimetics of phosphotyrosyl peptides having the formula R′Y*L, where R′ is a benzyl, pyridyl, or pyrazinyl derivative, including those set forth in FIG. 15, have also been shown to be STAT3 dimerization and DNA-binding inhibitors. ISS 610 is one such compound; see Turkson et al., Molecular Cancer Therapeutics, “Novel peptidomimetic inhibitors of signal transducer and activator of transcription 3 dimerization and biological activity,” 2004, p. 261-269.

An inhibitor of the Jak2/Stat3 pathway can inhibit Jak2. For example, an inhibitor of Jak2 can reduce the expression level of the Jak2 protein in a cell. Such an inhibitor can be an isolated nucleic acid that, when transcribed in a cell, results in an siRNA, a ribozyme, or an antisense nucleic acid. In other cases, an siRNA or antisense nucleic acid need not be transcribed in the cell, e.g., exogenous siRNA or antisense molecules for administration.

In some cases, the inhibitor of Jak2 inhibits an activity of Jak2. A Jak2 activity can be tyrosine kinase activity. Kinase activity can be evaluated as described previously. An inhibitor of Jak2 can bind noncovalently to Jak2. Noncovalent binding can be assessed using a number of analytical techniques well known to those of ordinary skill in the art, including competitive assays with known binders, surface plasmon resonance techniques, etc. In some cases, a noncovalent binder to Jak2 can be an antibody or antibody fragment, as discussed more fully below.

An inhibitor of Jak2 can be a small-molecule. A small-molecule inhibitor of Jak2 can bind noncovalently to Jak2. For example, AG490 is a small-molecule Jak2 inhibitor. Cucurbitacin Q (NSC 135075) is known to suppress Stat3 activation but not Jak2 activation; see Sun et al., Oncogene (2005): 1-10.

Certain inhibitors for use in the compositions and methods are not selective inhibitors for either the PI3k/Akt or Jak2/Stat3 pathways. Any of the following compounds can be used as inhibitors of either pathway: Herceptin (Trastuzamab); Erbitux (Cetuximab); Iressa (a small moleculeErbB 1 tyrosine kinase (EGFr) activity inhibitor; also known as gefitinib, having the chemical name N-(3-chloro-4-fluorophenyl)-7-methoxy-6-(3-morpholin-4-yl)-propoxy]quinazolin-4-amine)); Tarceva (a small-molecule EGFr blocker, erlotinib); Gleevec (imatinib mesylate, a bcr-abl tyrosine kinase inhibitor); and AG1478 (inhibitor of ErbB 1; chemical name 4-(3-Chloroanillino)-6,7-dimethoxyquinazoline).

Antibody and Antibody Fragment Inhibitors

An inhibitor can be an antibody or antibody fragment that is specific for a protein in a pathway described herein or for a protein that activates a pathway described herein. For example, antibodies or antibody fragments that exhibit specific binding affinity for Jak2, Stat3, PI3k, or Akt can be prepared and used in the described methods. In other cases, an antibody or antibody fragment that binds to a polypeptide that activates the Jak2/Stat3 pathway or PI3k/Akt pathway, or both, can be used. For example, an anti-ERbB1 monoclonal antibody, Cetuximab (Erbitux™, C225), can be used; an anti-ErbB2 monoclonal antibody, Trastuzamab (Herceptin) can be used; or a fully human anti-EGFr antibody, ABX-EGF (panitumumab) can be used.

Antibodies or antibody fragments for use herein are available commercially or can be prepared using methods known to those having ordinary skill in the art, as described herein and elsewhere. An antibody or antibody fragment includes a monoclonal antibody or antibody fragment, a humanized or chimeric antibody or antibody fragment, a single chain Fv antibody fragment, an Fab fragment, and an F(ab)₂ fragment. A chimeric antibody or antibody fragment is a molecule in which different portions are derived from different animal species, such as those having a variable region derived from a mouse monoclonal antibody and a human immunoglobulin constant region. Fully humanized antibodies or antibody fragments are also contemplated.

Monoclonal antibodies, which are homogeneous populations of antibodies to a particular antigenic epitope, can be prepared using standard hybridoma technology. In particular, monoclonal antibodies can be obtained by any technique that provides for the production of antibody molecules by continuous cell lines in culture such as described by Kohler et al., 1975, Nature, 256: 495, the human B-cell hybridoma technique (Kosbor et al., 1983, Immunology Today, 4: 72; Cole et al., 1983, Proc. Natl. Acad. Sci USA, 80: 2026), and the EBV-hybridoma technique (Cole et al., “Monoclonal Antibodies and Cancer Therapy,” Alan R. Liss, Inc., pp. 77-96 (1983). Such antibodies can be of any immunoglobulin class including IgG, IgM, IgE, IgA, IgD, and any subclass thereof. A hybridoma producing monoclonal antibodies can be cultivated in vitro and in vivo.

Antibody fragments that have a specific binding affinity can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Alternatively, Fab expression libraries can be constructed. See, for example, Huse et al., 1989, Science, 246: 1275. Once produced, antibodies or fragments thereof are tested for recognition of a particular polypeptide by standard immunoassay methods including ELISA techniques, radioimmunoassays and Western blotting. See, Short Protocols in Molecular Biology, Chapter 11, Green Publishing Associates and John Wiley & Sons, Edited by Ausubel, F. M. et al., 1992. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778. U.S. Pat. No. 6,303,341 discloses immunoglobulin receptors. U.S. Pat. No. 6,417,429 discloses immunoglobulin heavy- and light-chain polypeptides.

Inhibition via siRNA, Antisense, and Ribozymes

In some embodiments, an inhibitor can be an isolated nucleic acid. In some cases, an isolated nucleic acid can be an siRNA nucleic acid or an antisense nucleic acid, e.g., designed to be complementary to a target mRNA. For example, isolated double stranded siRNA nucleic acids and antisense nucleic acids can be chemically synthesized or produced via recombinant methods and purified. Such isolated nucleic acids can be contacted with a cell, e.g., delivered to a cell, and can result in an inhibition of gene expression. See the Examples below for an antisense and siRNA nucleic acid construct for Stat3.

In other cases, an inhibitor can be an isolated nucleic acid, such as a recombinant nucleic acid construct, that upon transformation and transcription in a cell, results in an RNA. Such an RNA can be useful for inhibiting expression of a gene, such as a gene encoding a protein in the pathways described herein or encoding a protein that activates one of the pathways described herein. For example, the expression of genes encoding Jak2, Stat3, Akt, or PI3k can be inhibited using isolated nucleic acids described herein.

Suitable nucleic acids from which such an RNA can be transcribed include antisense constructs. Thus, for example, a suitable nucleic acid can be an antisense nucleic acid construct to a target nucleic acid. As used herein, the term “target nucleic acid” refers to both RNA and DNA, including cDNA, genomic DNA, and synthetic (e.g., chemically synthesized) DNA. The target nucleic acid can be double-stranded or single-stranded (i.e., a sense or an antisense single strand). In some embodiments, the target nucleic acid encodes a polypeptide member of a pathway described herein, such as STAT3, JAK2, PI3k, or AKT. Thus, a “target nucleic acid” encompasses DNA encoding such a polypeptide, RNA (including pre-mRNA and mRNA) transcribed from such DNA, and also cDNA derived from such RNA.

An “antisense” compound is a compound containing nucleic acids or nucleic acid analogs that can specifically hybridize to a target nucleic acid, and the modulation of expression of a target nucleic acid by an antisense oligonucleotide is generally referred to as “antisense technology”. It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normnal function of the target nucleic acid, and (b) there is sufficient complementarity to avoid non-specific binding of the antisense oligonucleotide to non-target sequences under conditions in which specific binding is desired, i.e., under conditions in which in vitro assays are performed or under physiological conditions for in vivo assays or therapeutic uses.

Stringency conditions in vitro are dependent on temperature, time, and salt concentration (see, e.g., Sarmbrook et al., Molecular Cloning: A Laboratory Manual, Cold Spring Harbor Laboratory Press, NY (1989)). Typically, conditions of high to moderate stringency are used for specific hybridization in vitro, such that hybridization occurs between substantially similar nucleic acids, but not between dissimilar nucleic acids. Specific hybridization conditions are hybridization in 5× SSC (0.75 M sodium chloride/0.075 M sodium citrate) for 1 hour at 40° C. with shaking, followed by washing 10 times in 1× SSC at 40° C. and 5 times in 1× SSC at room temperature. Oligonucleotides that specifically hybridize to a target nucleic acid can be identified by recovering the oligonucleotides from the oligonucleotide/target hybridization duplexes (e.g., by boiling) and sequencing the recovered oligonucleotides.

In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions, such as those used in the RiboTAG™ technology described below. For example, hybridization can be carried out in vitro in 2× SSC (0.3 M sodium chloride/0.03 M sodium citrate), 0.1% SDS at 37° C. A wash solution containing 4× SSC, 0.1% SDS can be used at 37° C., with a final wash in 1× SSC at 45° C.

The specific hybridization of an antisense molecule with its target nucleic acid can interfere with the normal function of the target nucleic acid. For a target DNA nucleic acid, antisense technology can disrupt replication and transcription. For a target RNA nucleic acid, antisense technology can disrupt, for example, translocation of the RNA to the site of protein translation, translation of protein from the RNA, splicing of the RNA to yield one or more mRNA species, and catalytic activity of the RNA. The overall effect of such interference with target nucleic acid function is, in the case of a nucleic acid encoding a polypeptide in a pathway described herein, modulation of the expression of such a polypeptide. In the context of the present invention, “modulation” means a decrease in the expression of a gene (e.g., due to inhibition of transcription) and/or a decrease in cellular levels of the protein (e.g., due to inhibition of translation).

Antisense oligonucleotides are preferably directed at specific targets within a nucleic acid molecule. The process of “targeting” an antisense oligonucleotide to a particular nucleic acid usually begins with the identification of a nucleic acid sequence whose function is to be modulated. This nucleic acid sequence can be, for example, a gene (or mRNA transcribed from the gene) whose expression is associated with activation of the pathways described herein.

The targeting process also includes the identification of a site or sites within the target nucleic acid molecule where an antisense interaction can occur such that the desired effect, e.g., modulation of expression, will result. Traditionally, preferred target sites for antisense oligonucleotides have included the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions.

For maximal effectiveness, antisense oligonucleotides can be directed to regions of a target mRNA that are most accessible, i.e., regions at or near the surface of a folded mRNA molecule. Accessible regions of an mRNA molecule can be identified by methods known in the art, including the use of RiboTAG™ technology. This technology is disclosed in PCT application number SE01/02054. In the RiboTAG™ method, also known as mRNA Accessible Site Tagging (MAST), oligonucleotides that can interact with a test mRNA in its native state (i.e., under physiological conditions) are selected and sequenced, thus leading to the identification of regions within the test mRNA that are accessible to antisense molecules. In a version of the RiboTAG™ protocol, the test mRNA is produced by in vitro transcription and is then immobilized, for example by covalent or non-covalent attachment to a bead or a surface (e.g., a magnetic bead). The immobilized test mRNA is then contacted by a population of oligonucleotides, wherein a portion of each oligonucleotide contains a different, random sequence. Oligonucleotides that can hybridize to the test mRNA under conditions of low stringency are separated from the remainder of the population (e.g., by precipitation of the magnetic beads). The selected oligonucleotides then can be amplified and sequenced; these steps of the protocol are facilitated if the random sequences within each oligonucleotide are flanked on one or both sides by known sequences that can serve as primer binding sites for PCR amplification.

In general, oligonucleotides that are useful in RiboTAG™ technology contain between 15 and 18 random bases, flanked on either side by known sequences. These oligonucleotides are contacted by the test mRNA under conditions that do not disrupt the native structure of the mRNA (e.g., in the presence of medium pH buffering, salts that modulate annealing, and detergents and/or carrier molecules that minimize non-specific interactions). Typically, hybridization is carried out at 37 to 40° C., in a solution containing 1× to 5× SSC and about 0.1% SDS. Non-specific interactions can be minimized further by blocking the known sequence(s) in each oligonucleotide with the primers that will be used for PCR amplification of the selected oligonucleotides.

Alternatively, the transcription product of a nucleic acid can be similar or identical to the sense coding sequence of a sequence of interest, but is an RNA that is unpolyadenylated, lacks a 5′ cap structure, or contains an unsplicable intron. In some embodiments, the nucleic acid is a partial or full-length coding sequence that, in sense orientation results in inhibition of the expression of an endogenous polypeptide by co-suppression. Methods of co-suppression using a full-length cDNA sequence as well as a partial cDNA sequence are known in the art. See, e.g., U.S. Pat. No. 5,231,020.

In some cases, a nucleic acid can be transcribed into a ribozyme that affects expression of an mRNA, such as an mRNA encoding Jak2, Stat3, Akt, or PI3k. See U.S. Pat. No. 6,423,885. In general, a ribozyme is a catalytic RNA molecule that cleaves RNA in a sequence specific manner. Ribozymes that cleave themselves are called cis-acting ribozymes, while ribozymes that cleave other RNA molecules are called trans-acting ribozymes. Isolated nucleic acids can encode ribozymes designed to cleave particular mRNA transcripts, thus preventing expression of a polypeptide. A ribozyme sequence can have a sequence from a hammerhead, axhead, or hairpin ribozyme, and may be modified to have either slow cleavage activity or enhanced cleavage activity. For example, nucleotide substitutions can be made to modify cleavage activity as described elsewhere (see, e.g., Doudna and Cech, Nature, 418:222-228 (2002)). Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678. Hammerhead ribozyme sequences can be embedded in a stable RNA such as a transfer RNA (tRNA) to increase cleavage efficiency in vivo. Perriman, R. et al., Proc. Natl. Acad. Sci. USA, 92(13):6175-6179 (1995); de Feyter, R. and Gaudron, J., Methods in Molecular Biology, Vol. 74, Chapter 43, “Expressing Ribozymes in Plants”, Edited by Turner, P. C, Humana Press Inc., Totowa, N.J. RNA endoribonucleases such as the one that occurs naturally in Tetrahymena thermophila, and which have been described extensively by Cech and collaborators can be useful. See, for example, U.S. Pat. No. 4,987,071.

A suitable nucleic acid also can be transcribed into an interfering RNA. RNA interference, also known as gene silencing, typically employs small RNA molecules, called small interfering RNAs (siRNAs), to down-regulate the expression of targeted sequences in cells. siRNAs are double stranded molecules, one strand of which can be complementary to an mRNA. When an siRNA contains a sequence complementary to an mRNA, that mRNA is post-transcriptionally degraded by an RNA-Induced Silencing Complex (RISC) present within the cell (Hannon et al., Nature, 404:293-296 (2000)), thus effectively down-regulating expression of the associated gene. Thus siRNAs can be used to reduce the level of RNA (e.g., mRNA) within a cell.

Such an interfering RNA can be one that can anneal to itself, e.g., a double stranded RNA having a stem-loop structure: One strand of the stem portion of a double stranded RNA can comprise a sequence that is similar or identical to the sense coding sequence of an endogenous polypeptide, and that is from about 10 nucleotides to about 2,500 nucleotides in length. The length of the nucleic acid sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA can comprise an antisense sequence of an endogenous polypeptide, and can have a length that is shorter, the same as, or longer than the length of the corresponding sense sequence. The loop portion of a double stranded RNA can be from 10 nucleotides to 500 nucleotides in length, e.g., from 15 nucleotides to 100 nucleotides, from 20 nucleotides to 300 nucleotides, or from 25 nucleotides to 400 nucleotides in length. The loop portion of the RNA can include an intron. See, e.g., WO 98/53083; WO 99/32619; WO 98/36083; WO 99/53050; and US patent publications 20040214330 and 20030180945. See also, U.S. Pat. Nos. 5,034,323; 6,452,067; 6,777,588; 6,573,099; and U.S. Pat. No. 6,326,527.

Common molecular cloning and chemical nucleic acid synthesis techniques can be used to prepare isolated nucleic acids useful in the production of siRNAs, antisense molecules, and ribozymes for use in the methods. For example, PCR can be used to obtain a sense or antisense nucleic acid sequence, a ribozyme sequence, or an siRNA sequence. PCR refers to procedures in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complementary DNA strands. In addition, mutagenesis (e.g., site-directed mutagenesis) can be used to obtain components of the isolated nucleic acids provided herein. For example, site-directed mutagenesis can be used to design particular sense and antisense sequences within a nucleic acid construct.

Nucleic Acid Delivery

As described herein, any method can be used to deliver an isolated nucleic acid to a cell. In some embodiments, delivery of an isolated nucleic acid provided herein can be performed via biologic or abiologic means as described in, for example, U.S. Pat. No. 6,271,359. Abiologic delivery can be accomplished by a variety of methods including, without limitation, (1) loading liposomes with an isolated nucleic acid provided herein and (2) complexing an isolated nucleic acid with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene (Qiagen, Valencia, Calif.).

In some embodiments, systemic delivery is optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am Soc. Nephrol. 7: 1728 (1996)). Additional information regarding the use of liposomes to deliver isolated nucleic acids can be found in U.S. Pat. No. 6,271,359.

Pharmaceutical compositions containing the antisense oligonucleotides of the present invention also can incorporate penetration enhancers that promote the efficient delivery of nucleic acids, particularly oligonucleotides, to the skin. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

The mode of delivery can vary with the targeted cell or tissue. For example, isolated nucleic acids can be delivered to lung and liver tissue to treat a disease (e.g., cancer) via the intravenous injection of liposomes since both lung and liver tissue take up liposomes in vivo. In addition, when treating localized conditions such as cancer, catheritization in an artery upstream of the affected organ can be used to deliver liposomes containing an isolated nucleic acid. This catheritization can avoid clearance of the liposomes from the blood by the lungs and/or liver. For lesions such as skin cancer, human papilloma virus lesions, herpes lesions, and precancerous cervical dysplasia, topical delivery of liposomes can be used. Leukemias can be treated by ex vivo administration of the liposomes to, for example, to bone marrow.

Liposomes containing an isolated nucleic acid provided herein can be administered parenterally, intravenously, intramuscularly, intraperitoneally, transdermally, excorporeally, or topically. The dosage can vary depending on the species, age, weight, condition of the subject, and the particular compound delivered.

In other embodiments, biologic delivery vehicles can be used. For example, viral vectors can be used to deliver an isolated nucleic acid to a desired target cell. Standard molecular biology techniques can be used to introduce one or more of the isolated nucleic acids provided herein into one of the many different viral vectors previously developed to deliver nucleic acid to particular cells. These resulting viral vectors can be used to deliver the one or more isolated nucleic acids to the targeted cells by, for example, infection.

Methods for Treating, Preventing, or Ameliorating a Symptom of a Disease

The compositions and articles of manufacture described herein inhibit pathways associated with cancer and angiogenesis. The compositions therefore can find use in preventing, treating, or ameliorating one or more symptoms of cancer, such as solid or hematological cancers, and one or more symptoms of proliferative angiopathies, among other uses.

A method for treating, preventing, or ameliorating one or more symptoms of cancer in a mammal can include administering to the mammal:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof.

A mammal can be any mammal, including a human, dog, cat, monkey, rat, mouse, bird, sheep, horse, cow, or pig. A cancer can be a solid or hematological cancer, such as breast, prostate, melanoma, multiple myeloma, leukemia, pancreatic, ovarian, head and neck, and brain cancers. Any of the inhibitors described previously can be used. Any combination of such inhibitors can be used. Administration can be in any order and in any relative time frame. Typically, both inhibitors will be administered within about a 48 hour time frame, e.g., within about 36 hours, 24 hours, 18 hours, 12 hours, 8 hours, 4 hours, 2 hours, 1 hour, or simultaneously. The two inhibitors can be administered via the same or different routes of administration.

In some cases, an inhibitor of the Jak2/Stat3 signaling pathway and an inhibitor of the PI3k/Akt signaling pathway are capable of acting synergistically to treat, prevent, or ameliorate the one or more symptoms as compared to either inhibitor alone. Synergism can be evaluated, e.g., using in vitro assays or in vivo assays; see the Examples, below.

Provided also herein is a method for treating, preventing, or ameliorating one or more symptoms of a proliferative angiopathy in a mammal, which includes administering to the mammal:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof. The proliferative angiopathy can be diabetic microangiopathy. Any of the inhibitors described previously can be used. Any combination of such inhibitors can be used. Administration can be in any order and in any relative time frame. Typically, both inhibitors will be administered within about a 48 hour time frame, e.g., within about 36 hours, 24 hours, 18 hours, 12 hours, 8 hours, 4 hours, 2 hours, 1 hour, or simultaneously. The two inhibitors can be administered via the same or different routes of administration.

A method for inhibiting the growth of a cancer cell is also provided herein. The method can include contacting the cancer cell with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof. Any of the inhibitors described previously can be used. Any combination of such inhibitors can be used. Contacting of the cell with such inhibitors can be in any order and in any relative time frame. Typically, both inhibitors will be contacted with the cell within about a 48 hour time frame, e.g., within about 36 hours, 24 hours, 18 hours, 12 hours, 8 hours, 4 hours, 2 hours, 1 hour, or simultaneously. The two inhibitors can be contacted with the cell via the same or different routes of contacting, e.g., biologic and abiologic delivery mechanisms. The inhibitor of the Jak2/Stat3 signaling pathway and the inhibitor of the PI3k/Akt signaling pathway can be capable of acting synergistically to inhibit the growth of the cancer cell as compared to either inhibitor alone.

Similar methods can be used for inducing apoptosis in a cancer cell. Such a method can include contacting a cancer cell with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof, as described previously. In some cases, the inhibitor of the Jak2/Stat3 signaling pathway and the inhibitor of the PI3k/Akt signaling pathway are capable of acting synergistically to induce apoptosis in the cancer cell as compared to either inhibitor alone.

A method of inhibiting angiogenesis from a cancer tumor is also provided. The method can include contacting the cancer tumor with:

(a) an inhibitor of the Jak2/Stat3 signaling pathway, or a pharmaceutically acceptable salt thereof; and

(b) an inhibitor of the PI3k/Akt signaling pathway, or a pharmaceutically acceptable salt thereof.

Any of the inhibitors described previously can be used. Any combination of such inhibitors can be used. Contacting with the tumor can be in any order and in any relative time frame. Typically, both inhibitors will be contacted within about a 48 hour time frame, e.g., within about 36 hours, 24 hours, 18 hours, 12 hours, 8 hours, 4 hours, 2 hours, 1 hour, or simultaneously. The two inhibitors can be contacted with the tumor via the same or different routes of administration.

Pharmaceutical Compositions and Articles of Manufacture Including Pharmaceutical Compositions

In any of the methods, a composition or pharmaceutical composition including a composition described herein can be administered to a mammal, e.g., a human. The composition or pharmaceutical composition can be administered in a therapeutically effective amount. A pharmaceutical composition can include a composition described herein and a pharmaceutically acceptable carrier. As used herein, pharmaceutical composition and therapeutic preparation can be used interchangeably. For example, a composition can be provided together with physiologically tolerable (or pharmaceutically acceptable) liquid, gel or solid carriers, diluents, adjuvants and excipients. Such pharmaceutical compositions can be prepared as sprays (e.g. intranasal aerosols) for topical use. They may also be prepared either as liquid solutions or suspensions, or in solid forms including respirable and nonrespirable dry powders. Oral formulations (e.g. for gastrointestinal administration) usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. A pharmaceutical composition can take the form of a solution, suspension, tablet, pill, capsule, sustained release formulation, or powder, and typically contain 1%-95% of active ingredient (e.g., 2%-70%, 5%-50%, or 10-80%).

A composition can be mixed with diluents or excipients that are physiologically tolerable and compatible. Suitable diluents and excipients are, for example, water, saline, dextrose, glycerol, or the like, and combinations thereof. In addition, if desired, a composition may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

A pharmaceutical composition can be administered to a mammal (e.g., a human, mouse, rat, cat, monkey, dog, horse, sheep, pig, or cow) at a therapeutically effective amount or dosage level. A therapeutically effective amount or dosage level of a composition can be a function of many variables, including the affinity of the inhibitor for the protein, any residual activity exhibited by competitive antagonists, the route of administration, the clinical condition of the patient, and whether the inhibitor is to be used for the prophylaxis or for the treatment of acute episodes.

Effective dosage levels can be determined experimentally, e.g., by initiating treatment at higher dosage levels and reducing the dosage level until relief from reaction is no longer obtained. Generally, therapeutic dosage levels will range from about 0.01-100 μg/kg of host body weight.

A composition or pharmaceutical composition may also be administered in combination with one or more further pharmacologically active substances e.g., other chemotherapeutic agents, anti-angiogenic agents, immunomodulating agents, etc. An anti-angiogenic agent can be any agent known to affect angiogenesis, and in certain cases can be an anti-VEGF antibody or antibody fragment, dopamine, an anti-endothelial adhesion receptor of integrin alpha v3 antibody, thalidomide, a thalidomide analog, a protein kinase C beta inhibitor, 2-methoxyestradiol, interferon alpha, and interleukin 12.

In some cases, an anti-VEGF antibody or antibody fragment, such as a monoclonal anti-VEGF antibody, can be used as an anti-angiogenic agent. While not being bound by any theory, it is believed that an anti-VEGF antibody can block the interaction of VEGF with blood vessel receptors, thereby inhibiting angiogenesis. Any anti-VEGF antibody can be used, including a monoclonal anti-VEGF antibody, an anti-VEGF antibody fragment, and a humanized version of an anti-VEGF antibody. Any method can be used to obtain such antibodies, including those described elsewhere (e.g., U.S. Pat. Nos. 6,344,339; 6,448,077; 6,676,941 and U.S. 2003/0118657).

Any type of a chemotherapeutic agent can be used, including for example, taxol, vinblastin, vincristine, acyclovir, tacrine, gemcitabine, paclitaxel, methotrexate, cisplatin, bleomycin, doxorubicin, and cyclophosphamide. Any combinations of such chemotherapeutic agents can be used. Any method for preparing chemotherapeutic agents can be used, including those described elsewhere.

In view of the therapeutic urgency attendant acute episodes, a composition may be intravenously infused or introduced immediately upon the development of symptoms. Prophylaxis can be suitably accomplished, in certain cases, by intramuscular or subcutaneous administration. In this regard, the compositions can be prepared as injectables, either as liquid solutions or suspensions; solid forms suitable for solution in, or suspension in, liquid prior to injection may also be prepared.

The following detailed examples are provided for illustration and are not to be considered as limiting the scope of the present disclosure.

### EXAMPLES

Materials and Methods

The following reagents were purchased from various companies as indicated: Interleukin-6 (BD Pharmingen); Cycloheximide (Calbiochem); G418 (Cellgro); Anti-VEGF monoclonal antibody (R&D); Anti-HIF-1α polyclonal antibody and Anti-βactin monoclonal antibody (Santa Cruz Biotechnology); Anti-HIF-1β monoclonal antibody (NOVUS Biologicals); Anti-Phospho-AKT (Cell Signaling). Anti-AKT1 monoclonal antibody was a kind gift from Dr. J. Cheng, University of South Florida College of Medicine. DMEM, penicillin, and streptomycin were purchased from Invitrogen (Carlsbad, Calif.). Fetal bovine serum, propidium iodide, MTT, trypan blue, RNase A and LY294002 (the specific inhibitor of PI3K) were obtained from Sigma-Aldrich (St. Louis, Mo.). JSI-124 (a selective JAK2/STAT-3 activation inhibitor) was obtained from the NCI Developmental Therapeutics Program web site. APO-DIRECT Kit for terminal deoxynucleotidyl transferase-mediated UTP nick-end labeling (TUNEL) staining was purchased from BD Pharmingen. Polyclonal antibody to BCI-X_{L} was obtained from Oncogene Research Products (Cambridge, Mass.).

Generation of Stat3 knockdown tumor cell lines and Stat3 knockout MEFs

MCF-7 breast cancer cells and A2058 melanoma cells were cultured in high-glucose RPMI 1640 supplemented with 10% FBS and penicillin-streptomycin. The Stat3 siRNA oligonucleotide,

AATTAAAAAAGTCAGGTTGCTGGTCAAATTCTCTTGAAATTTGACCAGCAAC CTGACTTCC (SEQ ID NO:2), was inserted into pSilencer 1.0-U6 siRNA expression vector (Ambion). To generate siRNA/Stat3 stable tumor cell clones, the siRNAStat3 expression vector was co-transfected with pcDNA3 into MCF-7 and A2058 cells using Lipofectamine (Invitrogen), followed by G418 (1 mg/ml) selection. MCF-7 and A2058 clones stably transfected with the empty psilencer/pcDNA3 was used as control. Primary MEFs were prepared from Stat3flox mice (kindly provided by Drs. S. Akira and K. Takeda of Osaka University, Japan). To generate Stat3−/− MEFs, MEFs prepared from Stat3flox mice were transduced with retroviral Cre vector, and selected with puromycin. Deletion of the Stat3 gene in a majority of the Cre-transduced cells was confirmed by PCR and Western blot analysis. Control Stat3+/+ MEFs were generated from Stat3flox mice, but the MEFs were transduced with a control empty retroviral vector. The MEFs were maintained in DMEM with 10% FBS and penicillin-streptomycin.

Western Blot Analysis

MCF-7 cells and MEFs were serum starved for 20 h in serum-free medium before exposure to IL-6 for 6 h. Fifty pg of nuclear or whole-cell extracts was used for Western blot analysis. HIF-1α rabbit polyclonal antibody (H-206) (1:500 dilution), HIF-1β mouse monoclonal antibody (1:1,500 dilution), AKT1 mouse monoclonal, anti-phospho-AKT rabbit polyclonal, anti-VEGF monoclonal antibody (1:1,000 dilution) were used for the Western blot analyses. Horseradish peroxidase-conjugated sheep anti-mouse and donkey anti-rabbit or anti-goat secondary antibodies were used at 1:2,000 and 1:5,000 dilutions, respectively. The signal was developed with SuperSignal West Pico Chemiluminescent Substrate (PIERCE).

Electrophoretic Mobility Shift Assay (EMSA)

Nuclear extracts (1-8 μg of total protein) were incubated with the ³²P-radiolabled hSEE (high-affinity Sis-Inducible Element) oligonucleotide probe. Protein-DNA complexes were resolved by 5% non-denatured polyacrylamide gel electrophoresis (PAGE) and specific STAT/DNA complexes were detected by autoradiography.

Northern Blot Analysis

TRIzol reagent (Invitrogen) was used to isolate total RNAs, which were fractionated by 1% agarose-formaldehyde gel electrophoresis, followed by transferring to nylon membranes and hybridization with ³²P-labeled human HIF-1α cDNA.

Pulse-Label Assays

MCF-7 tumor cells (2×10⁶) were plated in a 10-cm dish, starved for 20 h, then treated with 20 ng/ml IL-6 for 30 min in methionine-free DMEM. Before harvesting cells, [³⁵S]Met-Cys was added to final concentration of 0.3 mCi/ml and pulse-labeled for 20 to 40 min. Preparation of extracts and immunoprecipitation with HIF-1α antibody was carried out as described in Laughner et al., Mol. Cell Biol. 21:3995-4004 (2001).

Matrigel Assays

2×10⁶ MCF-7 tumor cells stably transfected with either an empty control vector or Stat3siRNA expression vector were suspended in 100 μl PBS and mixed with 0.5 ml of Matrigel (Collaborative Biochemical Products) on ice, followed by injection subcutaneously into the abdominal midline of nude nice. On day 5, Matrigel plugs were harvested for photography and assaying hemoglobin contents. Hemoglobin quantification was carried out by the Drabkin method. Briefly, after dissecting away all the surrounding tissue, Matrigel pellets were melted at 4° and assayed for hemoglobin content (Drabkin's reagent kit, Sigma).

Cell Culture and Extract Preparation

All human breast cancer cell lines used were obtained from American Type Culture Collection (Manassas, Va.) and were cultured in DMEM medium supplemented with 10% fetal calf serum, 100 units/ml of penicillin, and 100 μg/ml of streptomycin. All cells were maintained at 37° C. in a humidified incubator with an atmosphere of 5% CO₂. A whole cell extract was prepared from these cells. Briefly, cells were harvested, washed with PBS twice, and homogenized in a HEPES lysis buffer [30 mM HEPES (pH 7.5), 1% Triton X-100, 10% glycerol, 10 mM NaCl, 5 mM MgCl₂, 25 mM NaF, 1 mM EGTA, 2 mM Na₂VO₄ 10 μg/ml soybean trypsin inhibitor, 25 μg/ml leupeptin, 10 μg/ml aprotinin, 2 mM phenylmethylsulfonyl fluoride, and 6.4 mg/ml 2-nitrophenylphosphate] for 30 min at 4° C. After that, the lysates were centrifuged at 12,000 g for 15 min, and the supernatants were collected as whole cell extracts.

MTT Assay

MDA-MB-468, MDA-MB-23 1, MCF-7 cells were grown to 50% confluency in a 96-well plate. Triplicate wells of cells were then treated with different concentrations of drugs either alone or in combination for 60 h. At the end of treatment 100 μl of 1 mg/ml MTT dissolved in serum-free medium was added to the cell cultures, followed by a 2-h incubation at 37° C. After cells were crystallized, the medium was removed and DMSO (100 μl) was added to dissolve the metabolized MTT product. The absorbance was then measured on a Wallac Victor² 1420 Multilabel counter at 540 nm.

Trypan Blue Assay

The trypan blue dye exclusion assay was performed by mixing 20 μl of cell suspension with 20 μl of 0.4% trypan blue dye before injecting into a hemocytometer and counting. The number of cells that absorbed the dye and those that exclude the dye were counted, from which the percentage of nonviable cell number to total cell number was calculated.

TUNEL Assay

Terminal deoxynucleotidyl transferase-mediated nick end labeling (TUNEL) was used to determine the extent of DNA strand breaks. The assay was performed following manufacturer's instruction using the APO-Direct kit. In brief, the harvested cells were fixed in 1% paraformaldehyde for 15 min on ice, washed with PBS, and then fixed again in 70% ethanol at −20 ° C. overnight. The cells were then incubated in DNA labeling solution [containing terminal deoxynucleotidyl transferase (TdT) enzyme, fluorescein-conjugated dUTP and reaction buffer] for 90 min at 37° C. After removing the DNA labeling solution by rinsing cells with Rinsing Buffer, the cells were incubated with the Propidium Iodide/RNase A solution, incubated for 30 min at room temperature in the dark, and then analyzed by flow cytometry within 3 h of staining.

Flow Cytometry

Cell cycle analysis based on DNA content was performed as we described previously. At each time point, cells were harvested, counted, and washed twice with wash buffer (1 mg glucose per ml PBS). Cells (2×10⁶) were suspended in 0.5 ml PBS, fixed in 5 ml of 70% ethanol for over night at −20° C., centrifuged, washed once with PBS and resuspended again in 1 ml of propidium iodide staining solution (50 jig propidium iodide, 100 units RNase A and 1 mg glucose per ml PBS), and incubated at room temperature in the dark for 30 min. The cells were then analyzed with FACScan (Becton Dickinson Immunocytometry, Calif.), ModFit LT and WinMDI V.2.8 cell cycle analysis software (Verity Software; Topsham, Me.). The cell cycle distribution is shown as the percentage of cells containing G₁, S, G₂, and M DNA judged by propidium iodide staining.

Western Blot Analysis

Human breast cancer MDA-MB-468, MDA-MB-23 1 and MDA-MB-453 cells were treated with single or different concentrations of LY294002 and JSI-124 for a specific or different time periods. After that cells were harvested and lysated. Cell lysates (50 μg) were separated by an SDS-PAGE and electrophoretically transferred to a nitrocellulose membrane, followed by enhanced chemiluminescence Western blotting. The enhanced chemiluminescence (ECL) Western Blot analysis was performed using specific antibodies.

### Example 1

### Activation of IL-6 Receptor Induces HIF-1α Expression

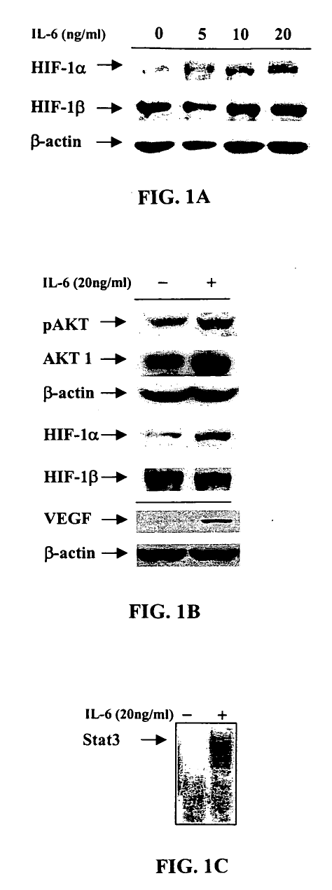

IL-6R signaling activates both JAK/Stat3 and PI3k/Aktsignaling pathways. To address whether IL-6R engagement activates HIF-1α increasing concentrations of IL-6 to MCF-7 human breast cancer cells were added. In the presence of increasing amounts of IL-6, HIF-1α protein levels in MCF-7 tumor cells were induced in a dose-dependent manner (FIG. 1A). Activation of IL-6R signaling in MCF-7 cells by IL-6 resulted in the activation of AKT and Stat3, as shown by phosphorylation of AKT (Western blot) and Stat3 DNA-binding (EMSA), respectively (FIG. 1B, C). Activation of these two signaling pathways coincides with an increase in VEGF protein expression in MCF-7 cells as well (FIG. 1B). In addition to the elevated level of phosphorylated AKT, the total protein level of AKT1 was also higher in MCF-7 cells treated with IL-6 (FIG. 1B).

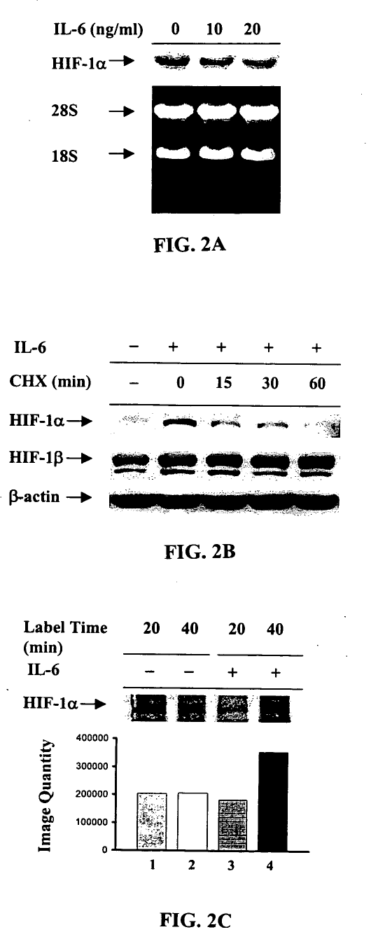

Northern blot analysis indicated that HIF-1α induction is not regulated at the mRNA level, but at the protein synthesis level, in MCF-7 breast cancer cells exposed to IL-6 (FIG. 2A). Blocking protein synthesis by cycloheximide (CHX) led to attenuation of HIF-1α expression induced by IL-6 (FIG. 2B). To confirm that IL-6R signaling-induced HIF-1α expression occurred at the protein synthesis level, [³⁵S]Met-Cys incorporation into HIF-1α protein was compared in MCF-7 tumor cells treated with either IL-6 or control medium (FIG. 2C). Results from these experiments established that IL-6 signaling affects HIF-1α expression at the protein synthesis level, consistent with the mechanism for growth signaling-induced HIF-1α regulation.

### Example 2

### Stat3 is Obligatory for IL-6-Induced HIF-1α and VEGF Expression

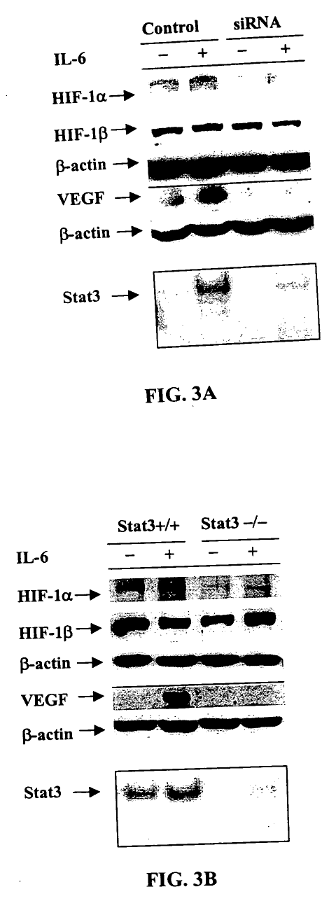

A previous study showed that in cervical cancer cells in which IL-6R signaling was constitutively activated, blocking Stat3 caused inhibition of VEGF expression. In contrast, targeting PI3K, which is expected to block AKT activation and thereby inhibit HIF-1α expression, did not interfere with VEGF expression. Considering the above results that IL-6 induced HIF-1α synthesis and previous findings that blocking Stat3 abrogated IL-6-induced VEGF upregulation, the role of Stat3 in HIF-1 expression was examined. To investigate whether Stat3 has a regulatory role in HIF-1α expression, HIF-1α induction by IL-6R signaling in tumor cells stably expressing siRNA/Stat3, an siRNA specific for Stat3 mRNA, was examined. MCF-7 tumor cells were transfected with either a control plasmid vector (pSilencer 1.0-U6) or the same vector encoding siRNA/Stat3. The effect of the siRNA inhibition of Stat3 in the tumor cells that survived G418 antibiotics selection was confirmed by Western blot analysis (data not shown) and by EMSA (FIG. 3A, bottom panel). Furthermore, while control cells exhibit detectable HIF-1α expression and an elevated level of HIF-1α upon IL-6 stimulation, little HIF-1α protein was detected in MCF-7 cells stably transfected with siRNA/Stat3, demonstrating the importance of Stat3 in basal level expression of HIF-1α (FIG. 3A, top panel). Moreover, whereas a significant induction of VEGF by IL-6 stimulation was observed in control MCF-7 cells, no VEGF expression was detectable in IL-6-treated MCF-7 cells expressing siRNA/Stat3 (FIG. 3A, top panel). These data suggest that Stat3 is necessary for both basal and IL-6-induced upregulation of HIF-1α and VEGF.

To rule out the possibility that MCF-7 tumor cells contain mutations that might affect the results described above, primary mouse embryonic fibroblasts (MEFs) were used to verify the findings. MEFs prepared from Stat3flox mice were transduced with either a control empty retroviral vector or retroviral vector encoding Cre recombinase. Those cells that express the Cre enzyme are expected to undergo Stat3 gene deletion. Stat3 DNA-binding activity was substantially reduced in Stat3flox MEFs transduced with Cre expression vector (FIG. 3B, bottom panel), indicating that the majority of the MEFs were transduced with the Cre-encoding virus and underwent deletion of the Stat3 alleles. In the Stat3−/− MEFs, IL-6-induced HIF-1α upregulation was markedly reduced (FIG. 3B, top panel), confirming the results with Stat3 siRNA-transfected MCF-7 tumor cells. Moreover, IL-6R signaling-mediated VEGF induction was not detectable under the experimental conditions in the Stat3−/− MEFs. Because IL-6R signaling activates both JAK/Stat3 and PI3k/Akt pathways, which are the main convergent pathways for numerous VEGF inducers, the data suggest that blocking Stat3 inhibits VEGF induction by a multitude of angiogenic inducers commonly activated in diverse cancers.

### Example 3

### Stat3 is Required for HIF-1α and VEGF Induction by Activated c-Src

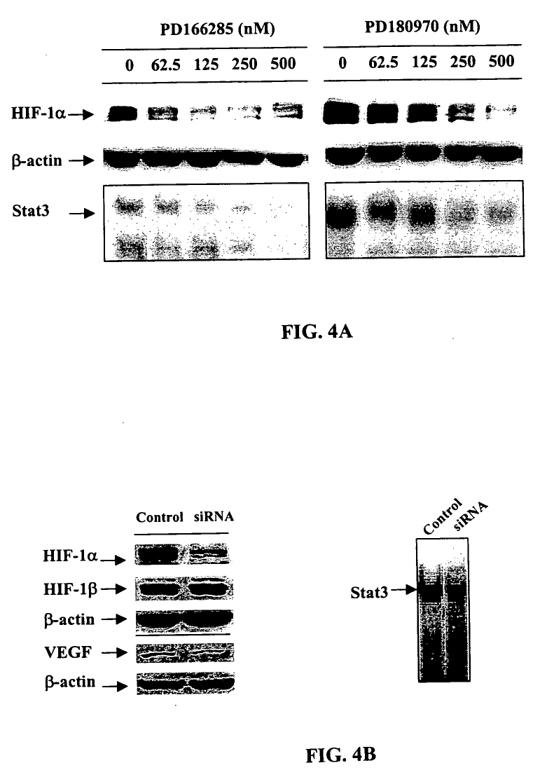

Like IL-6R signaling, Src tyrosine kinase is known to activate both JAK/Stat3 and PI3k/Aktpathways. Previous work has demonstrated that Src tyrosine kinase activity-induced VEGF expression requires Stat3, while other studies have shown that Src activity induces the protein synthesis of HIF-1α. The requirement for Stat3 in Src tyrosine kinase-induced HIF-1α expression in human A2056 melanoma cells was examined. It has been shown that c-Src is constitutively activated in these tumor cells, leading to persistent Stat3 activation. It has also been documented that blocking c-Src by two Src tyrosine kinase inhibitors reduces Stat3 activity in these tumor cells. After treating A2056 melanoma cells with PD166285 or PD180970 Src tyrosine kinase inhibitors, a dose-dependent reduction in HIF-1α protein level was observed (FIG. 4A, top panel). This was accompanied by a parallel reduction in Stat3 DNA-binding activity (FIG. 4A, bottom panel). To confirm that the reduction in HIF-1α expression by the Src inhibitors was due to inhibition of Stat3 signaling, the effects of siRNA/Stat3 on HIF-1α expression in these tumor cells were assessed. Interrupting Stat3 signaling in A2058 tumor cells by siRNA/Stat3 also reduced HIF-1α protein expression (FIG. 4B). Moreover, VEGF expression and Stat3 DNA-binding activity in A2058 melanoma cells were down-regulated in the presence of siRNA/Stat3 (FIG. 4B). These data demonstrate that blocking Stat3 signaling inhibits expression of both HIF-1α and VEGF induced by c-Src activity.

### Example 4

### Requirement of Stat3 Signaling for Her-2/Neu-Induced HIF-1α/VEGF Upregulation

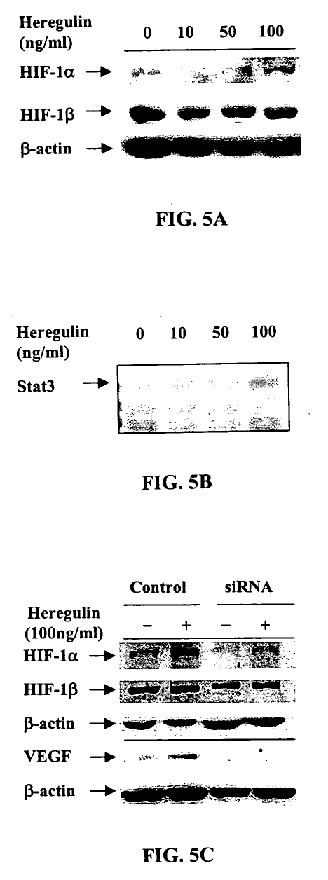

In addition to Src tyrosine kinase, activation of Her-2/Neu has also been shown to induce HIF-1α expression through the PI3k/Aktpathway. As shown in FIG. 5A, heregulin induces HIF-1α expression in MCF-7 cells. While MCF-7 breast cancer cells displayed little endogenous activated Stat3, stimulation with heregulin at 100 ng/ml led to detectable levels of activated Stat3 (FIG. 5B). This upregulation of Stat3 corresponded to an increase in HIF-lIa expression in control but not siRNA/Stat3 MCF-7 breast cancer cells (FIG. 5C). Moreover, Her-2 activation by heregulin upregulates VEGF expression in control but not Stat3/siRNA MCF-7 tumor cells, suggesting a critical requirement for Stat3 in Her-2-induced HIF-1α and VEGF expression.

Like Her-2, EGFR engagement/overactivity is known to activate Stat3 signaling. The results using a Stat3 antisense oligonucleotide (5′-AAAAAGTGCCCAGATTGCCC-3′, SEQ ID NO:1) indicate that Stat3 is also required for both HIF-1α and VEGF upregulation by EGF stimulation in DU145 human prostate cancer cells (data not shown).

### Example 6

### Effects of Small-Molecule Stat3 Inhibitors on HIF-1α and VEGF Expression

To date, several Stat3 inhibitors, such as a phosphopeptides, peptidomimetics, and platinum (IV) small-molecule complexes have been shown to inhibit Stat3 signaling with IC₅₀ values in the range of 5-250 μM. Moreover, these Stat3 inhibitors block Stat3-dependent malignant transformation and cell proliferation, and induce apoptosis of transformed mouse and human tumor cells displaying persistent Stat3 activity, with little or no effects on cells that are negative for this abnormality.

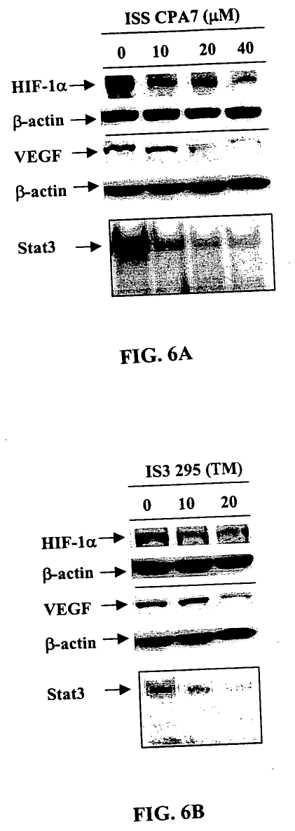

Small-molecule Stat3 inhibitors were evaluated for their ability to block HIF-1 and VEGF expression. Of the three tumor cell lines used in this study, A2058 and DU145 have relatively high Stat3 activity, whereas MCF-7 tumor cells do not. Treating DU145 cancer cells with either CPA-7 or IS3 295 platinum derivatives led to a reduction in Stat3 activity in a dose-dependent manner (FIG. 6A, B). Moreover, blocking Stat3 signaling in DU145 tumor cells by either Stat3 inhibitor caused a reduction in the expression of both HIF-1α and VEGF in the tumor cells. Inhibition of VEGF and HIF-1α expression in A2058 tumor cells treated with the Stat3 inhibitors was also observed (data not shown). These results provide evidence that molecular targeting of Stat3 with small molecule inhibitors is an effective approach to block tumor VEGF expression.

### Example 7

### Stat3 Regulates HIF-1α by Contributing to AKT Gene Expression

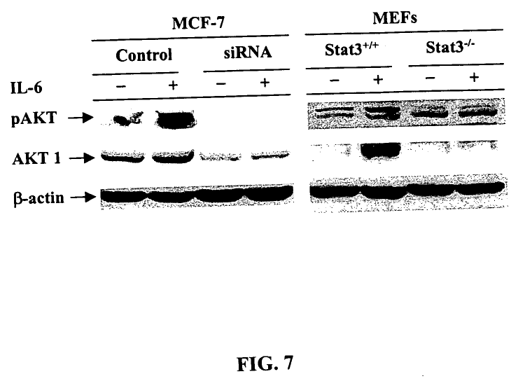

Stat3 is thus required for HIF-1α induction by IL-6R and other growth signaling molecules. The mechanism by which Stat3 regulates HIF-1α expression was therefore evaluated. Several reports have now established that HEF-1α induction by growth stimuli is mediated by the PI3k/Aktsignaling pathway. A search through a microarray gene expression database generated for the human breast cancer cell line, MDA-MB435, indicated that inhibition of Stat3 signaling by a Stat3 antisense oligonucleotide (5′ AAAAAGTGCCCAGATTGCCC-3′ (SEQ ID NO:1)) led to a reduction in AKT1 mRNA expression. Western blot analysis was performed to confirm that Stat3 is required for AKT1 expression and activity. IL-6 signaling-induced total AKT1 protein level was greatly reduced in Stat3 knockdown MCF-7 breast cancer cells (FIG. 7A, left panel). Moreover, AKT activity as indicated by levels of phosphorylated AKT was also lower in siRNA/Stat3 MCF-7 cells. To eliminate the possibility that tumor cells might have unique mutations that non-specifically influence these findings, the same experiments were performed using primary MEFs with or without the Stat3 alleles (FIG. 7A, right panel). Results from this set of experiments confirmed the microarray data that Stat3 is required for AKT1 expression, suggesting that Stat3 regulates HIF-1α levels through increasing AKT1 expression/activity.

### Example 8

### Stat3 is Required for Tumor Angiogenesis Induced by Both JAK/STAT and PI3k/Aktpathways

An evaluation of whether an inhibition of Stat3 would result in inhibition of tumor angiogenesis in vivo was performed. One interesting feature of targeting Stat3 for cancer therapy is that constitutive Stat3 activity in cancer cells is critical for tumor cell growth and survival, by virtue of Stat3's ability to upregulate anti-apoptotic genes such as Bc1-x_{L} and Mcl-1, and pro-proliferation genes including c-Myc and cyclin D½. This feature, however, also presents complications for Stat3-based anti-tumor angiogenesis assays, because targeting Stat3 by dominant-negative variant/mutants, antisense oligonucleotides and small-molecule inhibitors has been shown to cause tumor-specific growth inhibition/apoptosis. On the other hand, the present results indicate that siRNA/Stat3 transfected tumor cells in which Stat3 inhibition is not complete survive and grow well in short-term culture. Based upon these observations, siRNA/Stat3 tumor cells were used in an in vivo Matrigel assay, which is widely used for determining angiogenic capability. For the duration of the Matrigel assay, the proliferation rates of control and siRNA/Stat3 MCF-7 cells in culture were monitored. No difference in their growth rates was noted during the five days for completing the Matrigel assay in vivo (data not shown).

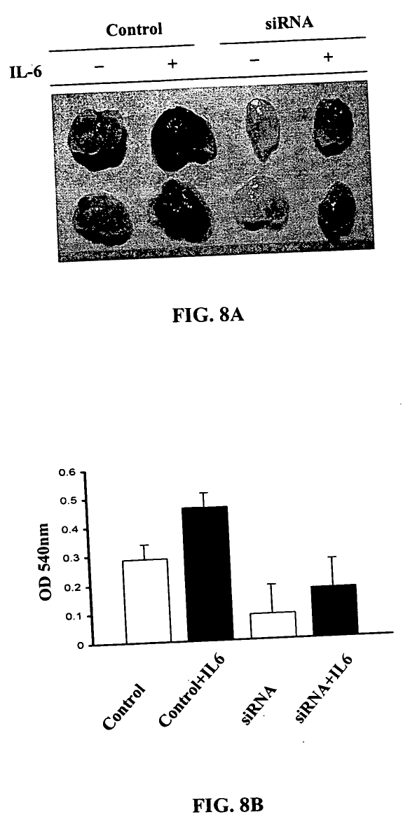

Because the results herein demonstrate that Stat3 is required for HIF-1α and VEGF upregulation mediated by both Jak2/Stat3 and PI3k/Akt pathways, Stat3 knockdown tumor cells can be predicted to have reduced tumor angiogenesis even when both signaling pathways are activated. To test this hypothesis, MCF-7 tumor cells stably transfected with either a control empty vector or siRNA/Stat3 expression vector were serum-starved for 4 h, followed by IL-6 stimulation to activate both Jak/Stat and PI3k/Akt pathways. The MCF-7 tumor cells were then mixed with Matrigel and implanted in vivo. Angiogenesis was considerably reduced in the Matrigel containing siRNA/Stat3 MCF-7 tumor cells compared to that of control MCF-7 cells (FIG. 8A). Moreover, when stimulated by IL-6, the control MCF-7 tumor cells were able to induce substantially more angiogenesis than their siRNA/Stat3 counterpart (FIG. 8A, B). These data show that blocking Stat3 signaling in tumor cells inhibits tumor angiogenesis induced by both Jak2/Stat3 and PI3k/Akt pathways. Because numerous oncogenic molecules depend on these two pathways for upregulating VEGF expression and angiogenesis, interrupting Stat3 signaling is expected to inhibit tumor angiogenesis stimulated by a multitude of VEGF inducers.

### Example 9

### Syneraistic Effect of Inhibition of Both Jak2/Stat3 and PI3k/Akt Pathways on Breast Cancer

Combined Inhibition of the Jak2/Stat3 and the PI3k/Akt Pathways is Synergistic for Inhibiting Breast Cancer Cell Growth/Proliferation

Several different pharmacological inhibitors were used to suppress constitutively activated PI3k/Akt and Jak2/Stat3 in breast cancer cells. The inhibitors used were LY294002 (a PI3k inhibitor) and JSI-124 ( a selective Jak2/Stat3 activation inhibitor).

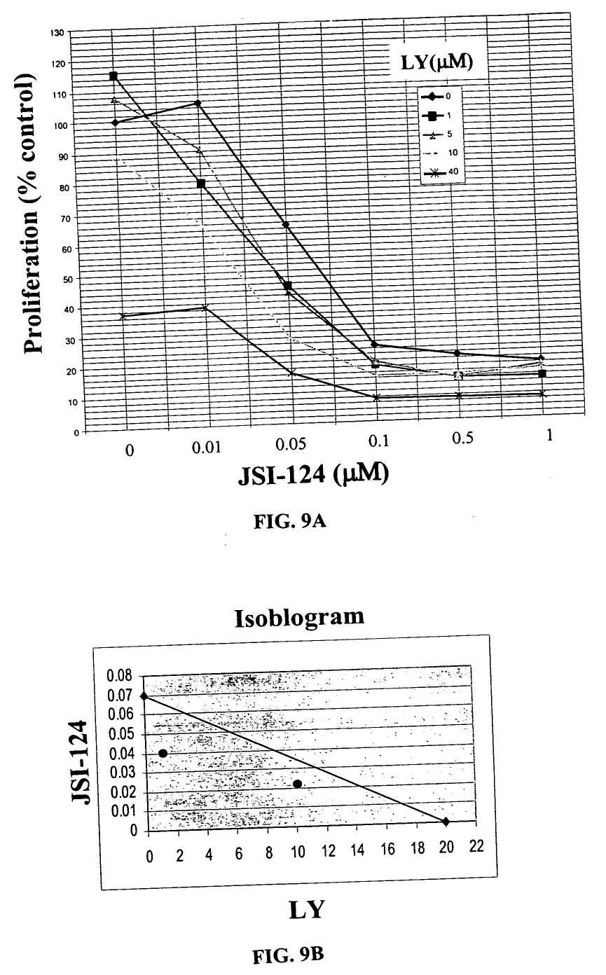

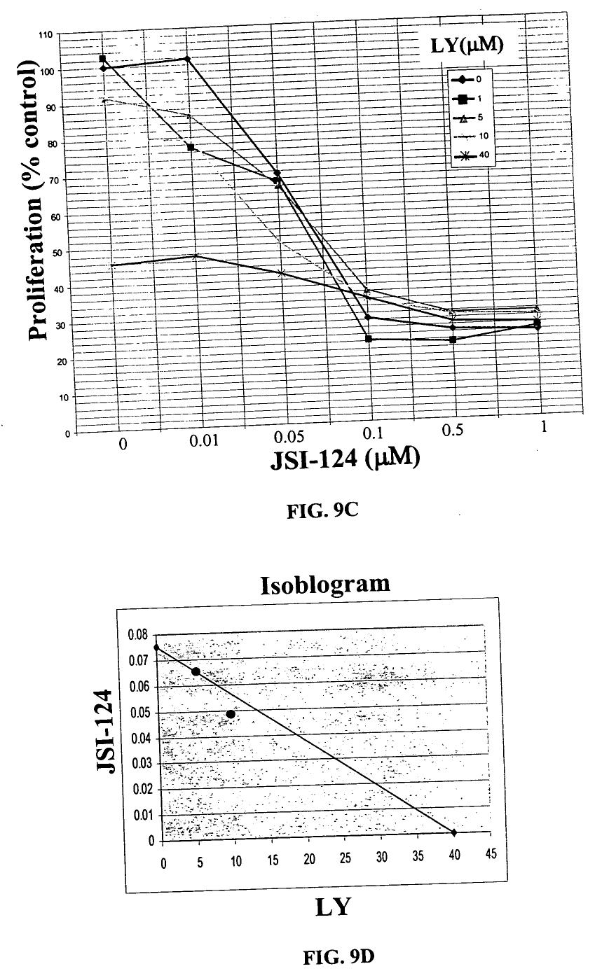

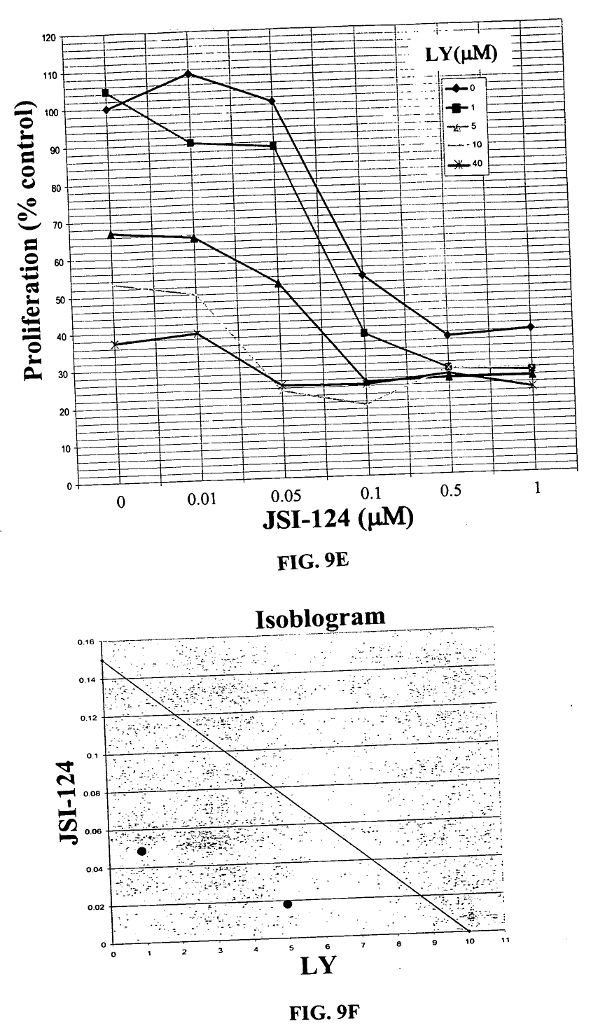

The above inhibitors were used alone or in combinations at different concentrations to treat different breast cancer cell lines (MDA-468, MDA-23 1, and MCF-7) for 60 h, followed by performance of MTT assay, which measures the status of cell viability and, thus, cell proliferation. The vehicle (DMSO) treated cells continued to proliferate after 60 h. After treatment, however, cellular proliferation decreased at a different rate with either drug alone or in combination in different cell lines. FIGS. 9A-9F show that the combination of LY294002 and JSI-124 result in synergistic effects in all three tested breast cancer cell lines.

Effect of LY294002, JSI-124 and their Combination on Breast Cancer Cell Death

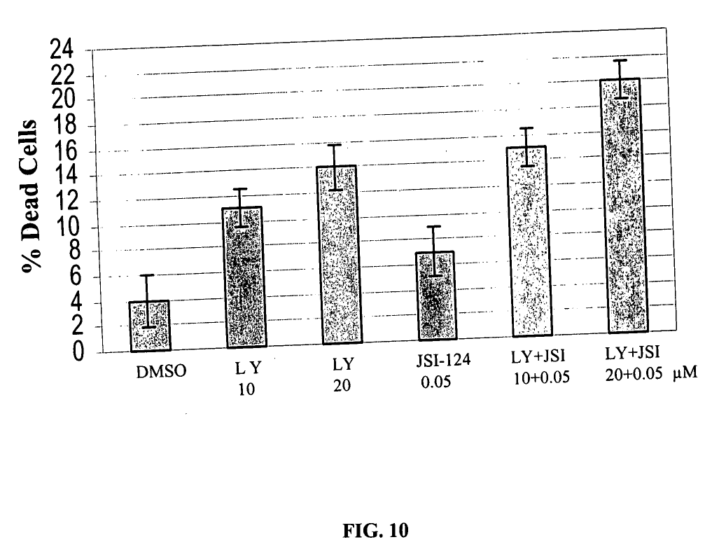

To determine whether the combination treatment is more beneficial than single agent treatment to induce breast cancer cell death, MDA-MB-468 cells were treated with the indicated concentrations of either drug alone or in combinations for 48 h, followed by trypan blue dye incorporation assays. Treatment of MDA-MB-468 cells with 20 μM LY294002+0.05 μM JSI-124 combination induced 20% cell death, whereas 20 μM LY294002 alone induced 14% cell death and 0.05 μM JSI-124 alone induced 7% cell death (FIG. 10). These results suggest that the combination treatment is additive at inducing breast cancer cell death.

LY294002 and JSI-124 Act Synergistically to Induce Apoptosis in Breast Cancer Cells

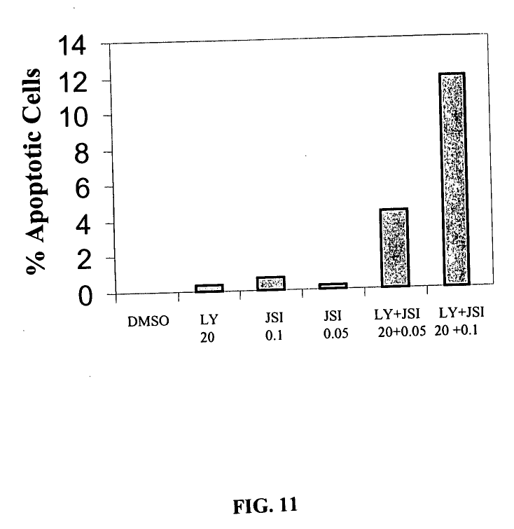

MDA-MB-468 cells were treated with the indicated concentrations of LY294002, JSI-124 either alone or in combination for 48 h, followed by TUNEL assays. Little apoptosis induction was observed when the drugs were used alone. In contrast, a total of 12% and 8% TUNEL-positive cells were observed when these 2 drugs were combined (FIG. 11). These results demonstrate that suppression of the Jak2/Stat3 and the PI3k/Akt pathways is synergistic at inducing apoptosis.

Combination Treatment of JSI and LY Results in Decreased Bc1XL Expression and Induction of PARP Cleavage in a Synergistic Manner

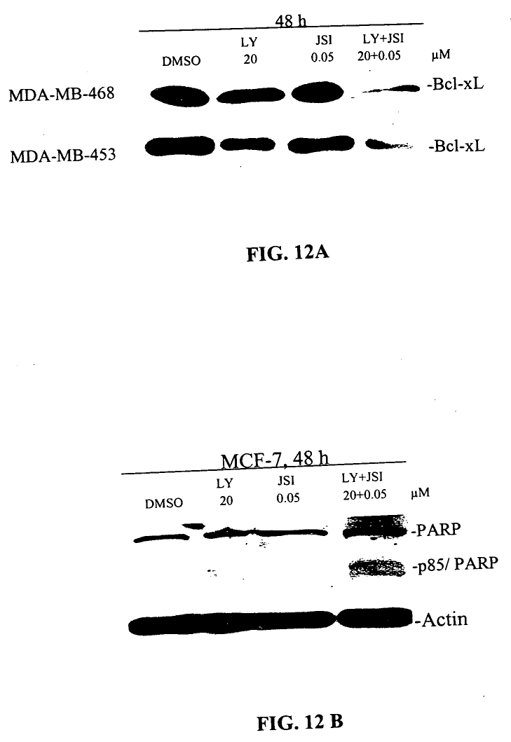

MDA-MB-468 and MDA-MB-453 breast cancer cells were treated with JSI or LY alone or in combination to determine the effects on the protein levels of the prosurvival protein Bc1XL. FIG. 12A shows that in both MDA-MB-468 and MDA-MB-453 cells, there was potent inhibition of Bc1-xL levels when the cells were treated with the drug combination but not with the single drugs treatment. This result suggest that down regulation of Bc1-xL is associated with the increased programmed cell death observed in FIG. 10.

Furthermore, when another breast cancer cell line, MCF-7, was treated with LY294002 and JSI-124, the combination treatment but not the single treatment, induced PARP cleavage (FIG. 12B).

Effect of Combination Treatment on Cell Cycle

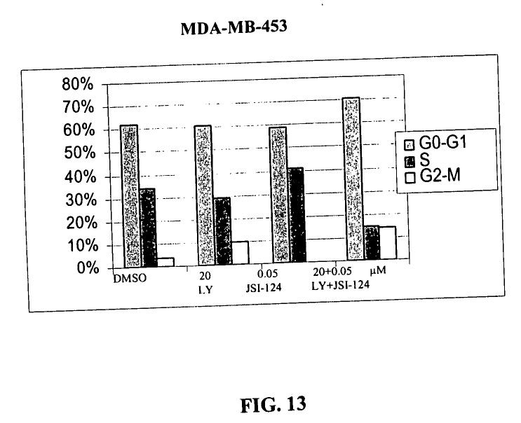

To determine the effect of combination treatment on cell cycle changes, MDA-MB-468 and MDA-MB453 cells were treated with the indicated concentrations of LY294002, JSI-124 or their combinations for 48 h, followed by flow cytometry analysis. FIG. 13 shows that combination treatment but not either single drug treatment resulted in accumulation in the G0/G1phase of the cell division cycle. Furthermore, the induction of G₀/G₁ phase accumulation was accompanied by a significant reduction in S phase cell population.

A number of embodiments of the invention have been described. Nevertheless, it will be understood that various modifications may be made without departing from the spirit and scope of the invention.

What is claimed is:

1. A composition of matter comprising:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof.

2. An article of manufacture comprising:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt 1 thereof.

3. The composition of claim 1, wherein said inhibitor of the Jak2/Stat3 signaling pathway inhibits a protein that activates Jak2.

4. The composition of claim 1, wherein said inhibitor of the Jak2/Stat3 signaling pathway does not inhibit the PI3k/Akt signaling pathway.

5. The composition of claim 1, wherein said inhibitor of the PI3k/Akt signaling pathway inhibits a protein that activates PI3k.

6. The composition of claim 1, wherein said inhibitor of the PI3k/Akt signaling pathway does not inhibit the Jak2/Stat3 signaling pathway.

7. The composition of claim 1, wherein said inhibitor of the Jak2/Stat3 signaling pathway inhibits Jak2.

8. The composition of claim 1, wherein said inhibitor of the Jak2/Stat3 signaling pathway inhibits Stat3.

9. The composition of claim 7, wherein said inhibitor of Jak2 reduces the expression level of the Jak2 protein in a cell.

10. The composition of claim 9, wherein said inhibitor of Jak2's expression level is an isolated nucleic acid that, when transcribed in a cell, results in an siRNA, a ribozyme, or an antisense nucleic acid.

11. The composition of claim 7, wherein said inhibitor of Jak2 inhibits an activity of Jak2.

12. The composition of claim 11, wherein said activity is a kinase activity.

13. The composition of claim 7, wherein said inhibitor of Jak2 binds noncovalently to Jak2.

14. The composition of claim 13, wherein said noncovalent binder to Jak2 is selected from an antibody or antibody fragment or a small molecule.

15. The composition of claim 8, wherein said inhibitor of Stat3 reduces the expression level of the Stat3 protein in a cell.

16. The composition of claim 15, wherein said inhibitor of Stat3's expression level is an isolated nucleic acid that, when transcribed in a cell, results in an siRNA, a ribozyme, or an antisense nucleic acid specific to the mRNA encoding Stat3.

17. The composition of claim 8, wherein said inhibitor of Stat3 inhibits an activity of Stat3.

18. The composition of claim 17, wherein said Stat3 activity is Stat3 dimerization, Stat3 DNA binding, or Stat3 transactivation.

19. The composition of claim 8, wherein said inhibitor of Stat3 binds noncovalently to Stat3.

20. The composition of claim 19, wherein said noncovalent binder to Stat3 is selected from an antibody or antibody fragment, or a small-molecule.

21. The composition of claim 20, wherein said small-molecule is CPA-1 or CPA-7.

22. The composition of claim 1, wherein said inhibitor of the PI3k/Akt pathway inhibits PI3k.

23. The composition of claim 22, wherein said inhibitor of PI3k reduces the expression level of the PI3k protein in a cell.

24. The composition of claim 23, wherein said inhibitor of PI3k's expression level is an isolated nucleic acid that, when transcribed in a cell, results in an siRNA, a ribozyme, or an antisense nucleic acid specific to the mRNA encoding PI3k.

25. The composition of claim 22, wherein said inhibitor of PI3k inhibits an activity of PI3k.

26. The composition of claim 25, wherein said PI3k activity is kinase activity.

27. The composition of claim 22, wherein said inhibitor of PI3k binds noncovalently to PI3k.

28. The composition of claim 27, wherein said noncovalent binder to PI3k is selected from an antibody or antibody fragment, or a small-molecule.

29. The composition of claim 1, wherein said inhibitor of the PI3k/Akt pathway inhibits Akt.

30. The composition of claim 29, wherein said inhibitor of Akt reduces the expression level of the Akt protein in a cell.

31. The composition of claim 30, wherein said inhibitor of Akt's expression level is an isolated nucleic acid that, when transcribed in a cell, results in an siRNA, a ribozyme, or an antisense nucleic acid specific to the mRNA encoding Akt.

32. The composition of claim 29, wherein said inhibitor of Akt inhibits an activity of Akt.

33. The composition of claim 32, wherein said Akt activity is kinase activity.

34. The composition of claim 29, wherein said inhibitor of Akt binds noncovalently to Akt.

35. The composition of claim 34, wherein said noncovalent binder to AKT is selected from an antibody or antibody fragment, or a small-molecule.

36. The composition of claim 35, wherein said small-molecule is TCN.

37. A pharmaceutical composition comprising the composition of claim 1, and a pharmaceutically acceptable carrier.

38. A composition of matter according to claim 1 for use in the treatment, prevention, or amelioration of one or more symptoms of cancer.

39. A pharmaceutical composition according to claim 37 for use in the treatment, prevention, or amelioration of one or more symptoms of cancer.

40. Use of a composition of claim 1 in the manufacture of a medicament for the therapeutic and/or prophylactic treatment of cancer.

41. An article of manufacture comprising:
(a) a pharmaceutical composition comprising an inhibitor of the Jak2/Stat3 signaling pathway, and a pharmaceutically acceptable carrier; and
(b) a pharmaceutical composition comprising an inhibitor of the PI3k/Akt signaling pathway, and a pharmaceutically acceptable carrier.

42. An article of manufacture according to claim 2 for use in the treatment, prevention, or amelioration of one or more symptoms of cancer.

43. An article of manufacture according to claim 41 for use in the treatment, prevention, or amelioration of one or more symptoms of cancer.

44. Use of an article of manufacture of claim 2 in the manufacture of a medicament for the therapeutic and/or prophylactic treatment of cancer.

45. A method for treating, preventing, or ameliorating one or more symptoms of cancer in a mammal, comprising administering:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof to said mammal.

46. The method of claim 45, wherein said mammal is a human.

47. The method of claim 45, wherein said cancer is selected from breast, prostate, melanoma, multiple myeloma, leukemia, pancreatic, ovarian, head and neck, and brain cancers.

48. The method of claim 45, wherein said inhibitor of the Jak2/Stat3 signaling pathway is an inhibitor of Stat3.

49. The method of claim 48, wherein said inhibitor of Stat3 is a small-molecule that binds noncovalently to Stat3.

50. The method of claim 49, wherein said small-molecule is CPA-I or CPA-7.

51. The method of claim 45, wherein said inhibitor of the PI3k/Akt signaling pathway is an inhibitor of PI3k.

52. The method of claim 51, wherein said inhibitor of PI3k is a small-molecule that binds noncovalently to PI3k.

53. The method of claim 45, wherein said inhibitor of the PI3k/Akt signaling pathway is an inhibitor of Akt.

54. The method of claim 53, wherein said inhibitor of Akt is a small-molecule that binds noncovalently to Akt.

55. The method of claim 54, wherein said small-molecule is TCN.

56. The method of claim 45, wherein said inhibitor of the Jak2/Stat3 signaling pathway and said inhibitor of the PI3k/Akt signaling pathway are capable of acting synergistically to treat, prevent, or ameliorate said one or more symptoms as compared to either inhibitor alone.

57. A method for treating, preventing, or ameliorating one or more symptoms of a proliferative angiopathy in a mammal, comprising administering to said mammal:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof.

58. The method of claim 57, wherein said proliferative angiopathy is diabetic microangiopathy.

59. A method for inhibiting the growth of a cancer cell comprising contacting said cancer cell with:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof; wherein said inhibitor of the Jak2/Stat3 signaling pathway and said inhibitor of the PI3k/Akt signaling pathway are capable of acting synergistically to inhibit said growth of said cancer cell as compared to either inhibitor alone.

60. A method for inducing apoptosis in a cancer cell comprising contacting said cancer cell with:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof; wherein said inhibitor of the Jak2/Stat3 signaling pathway and said inhibitor of the PI3k/Akt signaling pathway are capable of acting synergistically to induce apoptosis in said cancer cell as compared to either inhibitor alone.

61. A method of inhibiting angiogenesis from a cancer tumor, comprising contacting said cancer tumor with:
(a) an inhibitor of the Jak2/Stat3 signaling pathway, or a pharmaceutically acceptable salt thereof; and
(b) an inhibitor of the PI3k/Akt signaling pathway, or a pharmaceutically acceptable salt thereof.


SEQUENCE LISTING (6 sequences)

SEQ ID NO: 1 - DNA, 20 bp, Artificial Sequence
  Synthetically generated oligonucleotide
aaaaagtgcccagattgccc

SEQ ID NO: 2 - DNA, 61 bp, Artificial Sequence
  Synthetically generated oligonucleotide
aattaaaaaagtcaggttgctggtcaaattctcttgaaatttgaccagcaacctgacttcc

SEQ ID NO: 3 - protein, 6 aa, Artificial Sequence
  Exemplary motif
PYLKTK

SEQ ID NO: 4 - protein, 18 aa, Artificial Sequence
  Exemplary motif
PYLKTKAAVLLPVLLAAP

SEQ ID NO: 5 - protein, 3 aa, Artificial Sequence
  Exemplary motif
PYL

SEQ ID NO: 6 - protein, 3 aa, Artificial Sequence
  Exemplary motif
AYL